(19) United States
(12) Patent Application Publication
(10) Pub. No.: US 2007/0292400 A1
(43) Pub. Date: Dec. 20, 2007

(54) Media conditioned by human embryonic stem cells or other progenitor cells and uses therefor

(76) Inventors: Stuart A. Lipton, La Jolla, CA (US); Alexey Terskikh, La Jolla, CA (US)

Correspondence Address: BIOTECHNOLOGY LAW GROUP;C/O PORTFOLIOIP, PO BOX 52050, MINNEAPOLIS, MN, 55402, US

(21) Appl. No.: 11/707,367

(22) Filed: Feb. 15, 2007

Related U.S. Application Data
(60) Provisional application No. 60/774,765, filed on Feb. 16, 2006.

(51) Int. Cl. A61K 8/98; A61Q 19/00; A61Q 19/08

(52) U.S. Cl. 424/93.7

(57) ABSTRACT

Compositions for application to the skin of individuals in need thereof are provided that include media conditioned by the growth of human embryonic stem cells.

### CROSS-REFERENCE TO RELATED APPLICATIONS

This application claims priority from U.S. Provisional Application Ser. No. 60/774, 765 filed Feb. 16, 2006.

### FIELD OF THE INVENTION

The present invention relates to the field of cosmaceuticals. In particular, the present invention relates to compositions and methods for application to skin that include media conditioned by the growth of human embryonic stem cells.

### BACKGROUND

Skin is subject to a number of conditions that can lead to a desire for cosmetic enhancement. Such conditions include aging due to chronological aging or photoaging from exposure to the sun, or both. Skin aging results in wrinkling, the appearance of pigmented areas (“age spots”), thinning of the skin, loss of elasticity, and other undesirable characteristics. Other skin conditions that can be improved by cosmetic approaches include scarring, e.g., from acne or other causes, uneven pigmentation, and the like. A variety of procedures have been developed for improving skin appearance. Examples of such procedures include treatment with Botulinum Toxin Type A (“botox”), retinoids and derivatives thereof and especially retinoic acid (all-trans and 13-cis) and retinol, and the use of hydroxy acids. Although progress has been made in the use of cosmetic compositions for the treatment of skin, response to treatment is variable and often a condition is only marginally to moderately responsive to treatment. Unfortunately, once applied to the skin, some of these agents can cause itching, stinging and tightness which may lead to considerable discomfort. For many subjects, sensitivity to sun is enhanced. The use of these products by consumers with sensitive skin is often prevented. Many of the same considerations apply to other methods for cosmetic treatment of skin, such as laser resurfacing or dermabrasion. Thus, there remains a need for cosmetic and/or dermatological compositions and methods for the treatment of skin.

### SUMMARY OF THE INVENTION

The present invention provides skin-care compositions and methods involving the use of media conditioned by the growth of stem cells and other progenitor cells, including but not limited to human embryonic stem cells (hESC).

According to one embodiment of the invention, compositions are provided for administration to skin of an individual. Such compositions comprise an amount of a medium conditioned by growth of embryonic stem cells, including but not limited to human embryonic stem cells, that is effective to provide an improvement in the health or appearance of the skin of the individual and a suitable vehicle. The improvements in skin health or appearance that can be achieved through application of such compositions include but are not limited to improvements with respect to wrinkling, folds, sagging, age spots, uneven pigmentation, thinning, elasticity, scarring, surface roughness, surface roughness, surface vessels, redness, and pore size. According to another embodiment, such a composition is substantially free from non-human animal products.

According to another embodiment of the invention, methods are provided for topically treating skin of an individual in need thereof comprising administering to the skin of the individual any of the foregoing compositions.

According to another embodiment of the invention, methods are provided for making a composition for treatment of skin of an individual in need thereof comprising combining an amount of a medium conditioned by growth of embryonic stem cells, including but not limited to human embryonic stem cells, that is effective to provide an improvement in the appearance or health of the skin of the individual and a suitable vehicle. The resulting composition may be substantially free from non-human animal products.

Another embodiment of the invention provides for the use of a medium conditioned by growth of embryonic stem cells to prepare a medicament for administration to skin of an individual in need thereof.

According to another embodiment of the invention, kits are provided that comprise any of the above-mentioned compositions and suitable packaging.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### DETAILED DESCRIPTION OF THE INVENTION

The present invention provides various dermatological and cosmetic compositions for topical application to an individual that include medium conditioned by embryonic stem cells, including but not limited to human embryonic stem cells (hESC) or other progenitor cells, i.e., “conditioned medium,” and related compositions and methods.

According to one embodiment of the invention, the composition is formulated for application to the skin of an individual. The amount of conditioned medium in such a dermatological or cosmetic composition is an amount that is effective to provide a detectable improvement in the health or appearance of the skin of the treated individual, particularly at the site of application, as compared with untreated skin. Skin treatment with such compositions can result in improvement in conditions caused by or associated with injury or damage to the skin, such as aging due to chronological aging or photoaging from exposure to the sun, or both. Skin aging results in wrinkling, the appearance of pigmented areas (“age spots”), thinning of the skin, loss of elasticity, and other undesirable characteristics. Other skin conditions that can be improved by such treatment include scarring, e.g., from acne or other causes, uneven pigmentation, and the like. Such compositions can mitigate the effects of aging on skin, imparting one or more of the following benefits: skin rejuvenation benefits such as younger, healthier, radiant skin, even or non-blotchy texture tone and/or texture, removal or reduction of the appearance of such features as wrinkles or fine lines, surface roughness, folds or sagging (such as on the tissues of the cheeks, jowels, or brow), surface vessels, the number, size and pigmentation of age spots, redness, scars from acne or other sources, and reduction of pore size and appearance.

In certain embodiments, the compositions of the invention comprise lotions, creams, oils, gels, including hydrogels, powders, serums, salves, foundations, facial masks, lip care products, sunscreens, hair care products, such as shampoos, conditioners, including deep conditioners, hair care treatments, hot oil treatments, and the like, skin cleansers, exfoliants, compact formulations, or the like.

Definitions

The term “antioxidant” is used broadly to encompass any substance that inhibits or prevents oxidation or free radical formation. Thus, antioxidants include enzymes and other compounds that are able to counteract, at least in part, the damaging effects of free radicals produced by, among other things, ultraviolet light and environmental pollutants, in tissues such as, but not limited to, the skin. For example, the antioxidant defense system of the skin includes antioxidant enzymes and a group of low molecular weight antioxidants (LMWA). The LMWA have been shown to prevent oxidative damage, at least in part, by interacting with radical oxygen species, either directly or indirectly. Exemplary antioxidants are cysteine, glutathione, glutathione disulfide, glutathione peroxidase, glutathione reductase, catalase, vitamin E, including alpha- and gamma-tocopherol, ascorbic acid, ubiquinol 9, ubiquinone 9, and the like. Discussions of antioxidants may be found in, among other places, Kohen et al., Toxicology 148:149-157 (2000); Kohen, Biomed. Pharmacother. 53:181-192 (1999); Kohen et al., Methods of Enzymol. 300: 285-90, Academic Press (1999); Miyachi, Dermatol. Sci. 9:79-86 (1995); and Stohs, Basic Clin. Physio. Pharmacol. 6:206-228 (1995). The skilled artisan will understand that any and all culture-derived antioxidants in the conditioned media described herein are within the scope of the invention.

“Basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves can not synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12, although any base medium that can be supplemented with bFGF, insulin, and ascorbic acid and which supports the growth of primate primordial stem cells in a substantially undifferentiated state can be employed.

“Conditioned medium” refers to a growth medium that is further supplemented with soluble factors (“culture-derived growth factors”) derived from embryonic stem cells, preferably human embryonic stem cells, cultured in the medium. Techniques for isolating conditioned medium from a cell culture are well known in the art. As will be appreciated, conditioned medium is preferably essentially cell-free. In this context, “essentially cell-free” refers to a conditioned medium that contains fewer than about 10%, preferably fewer than about 5%, 1%, 0.1%, 0.01%, 0.001%, and 0.0001% than the number of cells per unit volume, as compared to the culture from which it was separated. As used herein, the term “conditioned medium” also encompasses medium conditioned by the growth of embryonic stem cells that that has been treated by concentration, extraction, or other means for preserving, increasing the potency, improving the stability, removing impurities, etc. Thus, conditioned medium includes extracts, for example, as defined below.

The term cosmeceutical refers to a formulation or composition comprising at least one biologically active ingredient that has an effect on the user of the product and at least one cosmeceutically-acceptable carrier. Cosmeceuticals may be viewed as cosmetics that, in certain applications and under appropriate conditions, may provide medicinal or drug-like benefits. In certain applications, for example, cosmeceuticals may affect the underlying structure of the skin, decrease wrinkle depth, or reverse or ameliorate the effect of photooxidation or aging on the skin. Cosmeceuticals may be particularly useful as skin care products, hair care products, and sun care products. In certain embodiments, cosmeceutical compositions comprise delivery systems including at least one of liposomes, cyclodextrins, polymer systems, or hyaluronic acid or related compounds. Cosmeceutical compositions comprise cosmeceutically-acceptable carriers. A pharmaceutically-acceptable carrier or formulation that is suitable for topical applications will typically also be a cosmeceutically-acceptable carrier or formulation. A topical cosmetic or cosmeceutical ointment, lotion, or gel composition typically contains a concentration of active ingredients comprising conditioned media or extracts thereof, from about 1 to 99%, about 5 to 95%, about 20 to 75%, or about 5 to 20%, in a cosmetically-acceptable carrier or a cosmeceutically-acceptable carrier, such as a pharmaceutical cream base, an oil-in-water emulsion, a water-in-oil emulsion, a gel, or the like. Various cosmetic and cosmeceutical compositions for topical use include drops, tinctures, lotions, creams, salves, serums, solutions, and ointments containing conditioned media or extracts, and an appropriate carrier, as discussed in greater detail below. The optimal percentage of the conditioned media or extract in each composition varies according to the composition's formulation and the therapeutic effect desired.

The term “culture-derived” refers to a component of conditioned cell culture media that is not present in the starting cell culture media that is used to culture and feed the cells, but is produced by the cultured cells and enters the media. Also within the meaning of the term culture-derived are compounds that are initially present in the pre-conditioned media, but whose concentration is increased during the culture process.

A “defined” medium refers to a biochemically defined formulation comprised solely of the biochemically-defined constituents. A defined medium may include solely constituents having known chemical compositions. A defined medium may also include constituents that are derived from known sources. For example, a defined medium may also include factors and other compositions secreted from known tissues or cells; however, the defined medium will not include the conditioned medium from a culture of such cells. Thus, a “defined medium” may, if indicated, include particular compounds added to form the culture medium, up to and including a portion of a conditioned medium that has been fractionated to remove at least one component detectable in a sample of the conditioned medium that has not been fractionated. Here, to “substantially remove” of one or more detectable components of a conditioned medium refers to the removal of at least an amount of the detectable, known component(s) from the conditioned medium so as to result in a fractionated conditioned medium that differs from an unfractionated conditioned medium in its ability to support the long-term substantially undifferentiated culture of primate stem cells. Fractionation of a conditioned medium can be performed by any method (or combination of methods) suitable to remove the detectable component(s), for example, gel filtration chromatography, affinity chromatography, immune precipitation, etc. Similarly, or a “defined medium” may include serum components derived from an animal, including human serum components. In this context, “known” refers to the knowledge of one of ordinary skill in the art with reference to the chemical composition or constituent. A defined medium for pluripotent stem cell culture is described in U.S. Patent Application 60/435,991 (published as 20070010011).

“Embryonic germ cells” or “EG cells” are cells derived from the primordial germ cells of an embryo or fetus that are destined to give rise to sperm or eggs. EG cells are among the embryonic stem cells that can be cultured in accordance with the invention.

“Embryonic stem cells” or “ES cells” are cells obtained from an animal (e.g., a primate, such as a human) embryo, preferably from an embryo that is less than about eight weeks old. Preferred embryonic stages for isolating primordial embryonic stem cells include the morula or blastocyst stage of a pre-implantation stage embryo. Well-known criteria for characterizing a cell as a stem cell are intended herein. See, e.g., Hoffman and Carpenter, Nature Biotech. 23:699-708, 2005, for a listing of such criteria.

“Extracellular matrix” (ECM) or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. The ECM thus encompasses essentially all secreted molecules that are immobilized outside of the cell. In vivo, the ECM provides order in the extracellular space and serves functions associated with establishing, separating, and maintaining differentiated tissues and organs. The ECM is a complex structure that is found, for example, in connective tissues and basement membranes, also referred to as the basal lamina. Connective tissue typically contains isolated cells surrounded by ECM that is naturally secreted by the cells. Components of the ECM have been shown to interact with and/or bind growth and differentiation factors, cytokines, matrix metalloproteases (MMPs), tissue inhibitors of metalloproteases (TIMPs), and other soluble factors that regulate cell proliferation, migration, and differentiation. Descriptions of the ECM and its components may be found in, among other places, Guidebook to the Extracellular Matrix, Anchor, and Adhesion Proteins, 2d ed., Kreis and Vale, eds., Oxford University Press, 1999 (“Kreis et al.”); Geiger et al., Nature Reviews Molecular Cell Biology 2:793-803, 2001; Iozzo, Annual Review of Biochemistry, 1998, Annual Reviews, Palo Alto, Calif.; Boudreau and Jones, Biochem. J. 339:481-88, 1999; Extracellular Matrix Protocols, Streuli and Grant, eds., Humana Press 2000; Metzler, Biochemistry the Chemical Reactions of Living Cells, 2d ed., vol. 1, 2001, Academic Press, San Diego, particularly chapter 8; and Lanza et al., particularly chapters 4 and 20.

Certain embodiments of the invention include at least one component of the ECM. In certain embodiments, the extracellular matrix component comprises: at least one protein, at least one glycoprotein, at least one proteoglycan, and at least one glycosaminoglycan. Exemplary glycoproteins, proteoglycans, and glycosaminoglycans include but are not limited to, collagen type I, collagen type II, collagen type III, collagen type IV, collagen type V, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, fibronectin, laminin, particularly laminin-1, laminin-2, laminin-4, and laminin-5, lumican, tenascin, versican, perlecan, thrombospondin, particularly thrombospondin-2 and thrombospondin-4, or laminin, particularly laminin-1, -2, -4, and -5, agrin, nidogen, bamacan, decorin, biglycan, fibromodulin, elastin, fibrillin, hyaluronan, vitronectin, chondroitin sulphate, dermatan sulphate, heparan sulphate, and keratan sulphate.

The term “extract” when used in reference to conditioned cell culture media refers to any subcomponent or fraction of the conditioned media, whether obtained by dialysis, fractionation, distillation, phase separation, gel filtration chromatography, affinity chromatography, hollow fiber filtration, precipitation, concentration, or the like.

“Feeder cells” are non-primordial stem cells on which stem cells, particularly primate primordial stem cells, may be plated and which provide a milieu conducive to the growth of the stem cells.

A cell culture is “essentially feeder-free” when it does not contain exogenously added conditioned medium taken from a culture of feeder cells nor exogenously added feeder cells in the culture, where “no exogenously added feeder cells” means that cells to develop a feeder cell layer have not been purposely introduced for that reason. Of course, if the cells to be cultured are derived from a seed culture that contained feeder cells, the incidental co-isolation and subsequent introduction into another culture of some small proportion of those feeder cells along with the desired cells (e.g., undifferentiated primate primordial stem cells) should not be deemed as an intentional introduction of feeder cells. Similarly, feeder cells or feeder-like cells that develop from stem cells seeded into the culture shall not be deemed to have been purposely introduced into the culture.

A “growth environment” is an environment in which stem cells (e.g., primate primordial stem cells) will proliferate in vitro. Features of the environment include the medium in which the cells are cultured, and a supporting structure (such as a substrate on a solid surface) if present.

The term “growth factor” as used herein refers to a protein, a polypeptide, or a complex of polypeptides, including cytokines, chemokines, morphogents, neutralizing antibodies, other proteins, and small molecules that are produced by a cell and which can affect itself and/or a variety of other neighboring or distant cells. Typically growth factors affect the growth and/or differentiation of specific types of cells, either developmentally or in response to a multitude of physiological or environmental stimuli. Some, but not all, growth factors are hormones. Culture-derived growth factors are preferred that have are not deleterious to the health and appearance of skin. Exemplary growth factors are insulin, insulin-like growth factor (IGF), nerve growth factor, VEGF, keratinocyte growth factor (KGF), fibroblast growth factors (FGFs), including basic FGF (bFGF), platelet-derived growth factors (PDGFs), including PDGF-AA and PDGF-AB, hepatocyte growth factor (HGF), transforming growth factor alpha (TGFα), transforming growth factor beta (TGFβ), including TGFβ₁ and TGF β₃, epidermal growth factor (EGF), granulocyte-macrophage colony-stimulating factor (GM-CSF), granulocyte colony-stimulating factor (G-CSF), interleukin-6 (IL-6), IL-8, activin-A, bone morphogenic proteins (BMPs) and the like. Growth factors are discussed in, among other places, Molecular Cell Biology, Scientific American Books, Darnell et al., eds., 1986; The Molecular and Cellular Biology of Wound Repair, Clark, Plenum Press, 1996; and Principles of Tissue Engineering, 2d ed., Lanza et al., eds., Academic Press, 2000. The skilled artisan will understand that any and all culture-derived growth factors in the conditioned media described herein are within the scope of the invention.

“Isotonic” refers to a solution having essentially the same tonicity (i.e., effective osmotic pressure equivalent) as another solution with which it is compared. In the context of cell culture, an “isotonic” medium is one in which cells can be cultured without an appreciable net flow of water across the cell membranes.

A solution having “low osmotic pressure” refers to a solution having an osmotic pressure of less than about 300 milli-osmols per kilogram (“mOsm/kg”).

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “non-essential amino acid” refers to an amino acid species that need not be added to a culture medium for a given cell type, typically because the cell synthesizes, or is capable of synthesizing, the particular amino acid species. While differing from species to species, non-essential amino acids are known to include L-alanine, L-asparagine, L-aspartic acid, L-glutamic acid, glycine, L-proline, and L-serine.

A “primate-derived primordial stem cell” or “primate primordial stem cell” is a primordial stem cell obtained from a primate species, including humans and monkeys, and includes genetically modified primordial stem cells.

“Pluripotent” refers to cells that are capable of differentiating into one of a plurality of different cell types, although not necessarily all cell types. An exemplary class of pluripotent cells is embryonic stem cells, which are capable of differentiating into any cell type in the human body. Thus, it will be recognized that while pluripotent cells can differentiate into multipotent cells and other more differentiated cell types, the process of reverse differentiation (i.e., de-differentiation) is likely more complicated and requires “re-programming” the cell to become more primitive, meaning that, after re-programming, it has the capacity to differentiate into more or different cell types than was possible prior to re-programming.

A cell culture is “essentially serum-free” when it does not contain exogenously added serum, where no “exogenously added feeder cells” means that serum has not been purposely introduced into the medium. Of course, if the cells being cultured produce some or all of the components of serum, of if the cells to be cultured are derived from a seed culture grown in a medium that contained serum, the incidental co-isolation and subsequent introduction into another culture of some small amount of serum (e.g., less than about 1%) should not be deemed as an intentional introduction of serum.

The term “skin” refers to the external skin surface of an individual. The uppermost layer of typical skin tissue is composed of dead cells which form a tough, horny protective coating. There is a thin outer layer, the epidermis and a thicker inner layer, the dermis. Intertwining S-like finger shaped portions are at the interface between the epidermal papillary layer and the dermal papillary layer, and extend downward. Beneath the dermis is the subcutaneous tissue, which often contains a significant amount of fat. The dermis layer contains the major part of the connective collagen, though viable collagen connective tissue also exists to a certain degree in the lower subcutaneous layer as well. Other structures found in typical skin include hair and associated follicles, sweat or sebaceous glands and associated pores, blood vessels and nerves. Additionally, a pigment layer might be present.

“Stem cell” includes any stem or progenitor cell, whether from a human or non-human source, and cells derived from stem cells that retain characteristics of progenitor cells.

The term “substantially free from,” refers to conditioned media or extracts thereof that contain little to no of a particular substance. In certain embodiments, the conditioned cell culture media comprises less than 49.999%, 30%, 20%, 10%, 5%, 1%, 0.5%, 0.05%, or no (0%) phenol red. In certain embodiments, the conditioned media comprises less than 49.999%, 30%, 20%, 10%, 5%, 1%, 0.5%, 0.05%, or no (0%) components of bovine-origin. Exemplary media components of bovine-origin include fetal calf serum, calf serum, bovine serum, bovine collagen, bovine insulin, bovine transferrin, and the like. In certain embodiments, the conditioned media comprises less than 49.999%, 30%, 20%, 10%, 5%, 1%, 0.5%, 0.05%, or no (0%) non-human animal products. In addition to the exemplary components of bovine-origin, listed above, non-human animal products include any animal products not of human origin, such as tissue culture components and products of porcine-origin. The skilled artisan will know that “serum-free” media and animal product-free media are commercially available from several vendors of cell culture media. Likewise, phenol red free media is also commercially available or can be prepared.

“Substantially undifferentiated” means that population of stem cells (e.g., primate primordial stem cells) contains at least about 50%, preferably at least about 60%, 70%, or 80%, and even more preferably, at least about 90%, undifferentiated, stem cells. Fluorescence-activated cell sorting using labeled antibodies or reporter genes/proteins (e.g., enhanced green fluorescence protein [EGFP]) to one or more markers indicative of a desired undifferentiated state (e.g., a primordial state) can be used to determine how many cells of a given stem cell population are undifferentiated. For purposes of making this assessment, one or more of cell surface markers correlated with an undifferentiated state (e.g., Oct-4, SSEA-4, Tra-1-60, and Tra-1-81) can be detected. Telomerase reverse transcriptase (TERT) activity and alkaline phosphatase can also be assayed. In the context of primate primordial stem cells, positive and/or negative selection can be used to detect, for example, by immuno-staining or employing a reporter gene (e.g., EGFP), the expression (or lack thereof) of certain markers (e.g., Oct-4, SSEA4, Tra-1-60, Tra-1-81, SSEA-1, SSEA-3, nestin, telomerase, Myc, p300, and Tip60 histone acetyltransferases, and alkaline phosphatase activity) or the presence of certain post-translational modifications (e.g., acetylated histones), thereby facilitating assessment of the state of self-renewal or differentiation of the cells.

“Totipotent” refers to cells that are capable of differentiating into any cell type, including pluripotent, multipotent, and fully differentiated cells (i.e., cells no longer capable of differentiation into various cell types), such as, without limitation, embryonic stem cells, neural stem cells, bone marrow stem cells, hematopoietic stem cells, cardiomyocytes, neuron, astrocytes, muscle cells, and connective tissue cells.

Primate-Derived Primordial Stem Cells

Stem cells, including primate primordial stem cells, cultured in accordance with the invention can be obtained from any suitable source using any appropriate technique. For example, procedures for isolating and growing human primordial stem cells are described in U.S. Pat. No. 6,090,622. Procedures for obtaining Rhesus monkey and other non-human primate primordial stem cells are described in U.S. Pat. No. 5,843,78 and international patent publication WO 96/22362. In addition, methods for isolating Rhesus monkey primordial stem cells are described by Thomson, et al., Proc. Natl. Acad. Sci. USA 92:7844-7848, 1995).

Human embryonic stem cells (hESCs) can be isolated, for example, from human blastocysts obtained from human in vivo preimplantation embryos, in vitro fertilized embryos, or one-cell human embryos expanded to the blastocyst stage (Bongso, et al. (1989), Hum. Reprod. 4:706). Human embryos can be cultured to the blastocyst stage in G1.2 and G2.2 medium (Gardner, et al. (1998), Fertil. Steril. 69:84). The zona pellucida is removed from blastocysts by brief exposure to pronase (Sigma). The inner cell masses can be isolated by immunosurgery or by mechanical separation, and are plated on mouse embryonic feeder layers, or in the defined culture system as described herein. After nine to fifteen days, inner cell mass-derived outgrowths are dissociated into clumps either by exposure to calcium and magnesium-free phosphate-buffered saline (PBS) with 1 mM EDTA, by exposure to dispase, collagenase, or trypsin, or by mechanical dissociation with a micropipette. The dissociated cells are then replated as before in fresh medium and observed for colony formation. Colonies demonstrating undifferentiated morphology are individually selected by micropipette, mechanically dissociated into clumps, and replated. Embryonic stem cell-like morphology is characterized as compact colonies with apparently high nucleus to cytoplasm ratio and prominent nucleoli. Resulting embryonic stem cells are then routinely split every 1-2 weeks by brief trypsinization, exposure to Dulbecco's PBS (without calcium or magnesium and with 2 mM EDTA), exposure to type IV collagenase (about 200 U/mL), or by selection of individual colonies by mechanical dissociation, for example, using a micropipette.

Once isolated, the stem cells, e.g., primate stem cells, can be cultured in a culture medium according to the invention that supports the substantially undifferentiated growth of primate primordial stem cells using any suitable cell culturing technique. For example, a matrix laid down prior to lysis of primate feeder cells (preferably allogeneic feeder cells) or a synthetic or purified matrix can be prepared using standard methods. The primate primordial stem cells to be cultured are then added atop the matrix along with the culture medium. In other embodiments, once isolated, undifferentiated human embryonic stem cells can be directly added to an extracellular matrix that contains laminin or a growth-arrested human feeder cell layer (e.g., a human foreskin fibroblast cell layer) and maintained in a serum-free growth environment according to the culture methods of invention. Unlike existing human embryonic stem cell lines cultured using conventional techniques, human embryonic stem cells and their derivatives prepared and cultured in accordance with the instant methods can be used therapeutically since they will not have been exposed to animal feeder cells, feeder-cell conditioned media, or serum at some point of their life time, thereby avoiding the risks of contaminating human cells with non-human animal cells, transmitting pathogens from non-human animal cells to human cells, forming heterogeneous fusion cells, and exposing human cells to toxic xenogeneic factors.

Alternatively, the stem cells, e.g., primate primordial stem cells, can be grown on living feeder cells (preferably allogeneic feeder cells) using methods known in the cell culture arts. The growth of the stem cells is then monitored to determine the degree to which they have become differentiated, for example, using a marker for alkaline phosphatase or telomerase or detecting the expression of the transcription factor Oct-4, or by detecting a cell surface marker indicative of an undifferentiated state (e.g., in the context of human embryonic stem cells, a labeled antibody for any one or more of SSEA-4, Tra-1-60, and Tra-1-81). When the culture has grown to confluence, at least a portion of the undifferentiated cells is passaged to another culture vessel. The determination to passage the cells and the techniques for accomplishing such passaging can be performed in accordance with the culture methods of invention (e.g., through morphology assessment and dissection procedures).

In certain preferred embodiments, the cells are cultured in a culture vessel that contains a substrate selected from the group consisting of feeder cells, preferably allogeneic feeder cells, an extracellular matrix, a suitable surface and a mixture of factors that adequately activate the signal transduction pathways required for undifferentiated growth, and a solution-borne matrix sufficient to support growth of the stem cells in solution. Thus, in addition to the components of the solution phase of culture media of the invention, the growth environment includes a substrate selected from the group consisting of primate feeder cells, preferably allogeneic feeder cells, and an extracellular matrix, particularly laminin. Preferred feeder cells for primate primordial stem cells include primate fibroblasts and stromal cells. In preferred embodiments, the feeder cells and stem cells are allogeneic. In the context of human embryonic stem cells, particularly preferred feeder cells include human fibroblasts, human stromal cells, and fibroblast-like cells derived from human embryonic stem cells. If living feeder cells are used, as opposed to a synthetic or purified extracellular matrix or a matrix prepared from lysed cells, the cells can be mitotically inactivated (e.g., by irradiation or chemically) to prevent their further growth during the culturing of primate primordial stem cells. Inactivation is preferably performed before seeding the cells into the culture vessel to be used. The primate primordial stem cells can then be grown on the plate in addition to the feeder cells. Alternatively, the feeder cells can be first grown to confluence and then inactivated to prevent their further growth. If desired, the feeder cells may be stored frozen in liquid nitrogen or at −140° C. prior to use. As mentioned, if desired such a feeder cell layer can be lysed using any suitable technique prior to the addition of the stem cells (e.g., primate stem cells) so as to leave only an extracellular matrix.

Not wishing to be bound to any theory, it is believed that the use of such feeder cells, or an extracellular matrix derived from feeder cells, provides one or more substances necessary to promote the growth of stem cells (e.g., primate primordial stem cells) and/or prevent or decrease the rate of differentiation of such cells. Such substances are believed to include membrane-bound and/or soluble cell products that are secreted into the surrounding medium by the feeder cells. Thus, those skilled in the art will recognize that additional cell lines can be used with the cell culture media of the present invention to equivalent effect, and that such additional cell lines can be identified using standard methods and materials, for example, by culturing over time (e.g., several passages) substantially undifferentiated primate primordial stem cells on such feeder cells in a culture medium according to the invention and determining whether the stem cells remain substantially undifferentiated over the course of the analysis. Also, because of the defined nature of the culture media provided herein, it is now possible to assay various compounds found in the extracellular matrix or secreted by feeder cells to determine their respective roles in the growth, maintenance, and differentiation of stem calls such as primate primordial stem cells.

When purified components from extracellular matrices are used in lieu of feeder cells, such components will include those provided by the extracellular matrix of a suitable feeder cell layer. Components of extracellular matrices that can be used include laminin, or products that contain laminin, such as Matrigel™, or other molecules that activate the laminin receptor and/or its downstream signaling pathway. Thus, for purposes of the invention, a molecule that activates the laminin receptor and/or its downstream signaling pathway in an analogous fashion to laminin (even with greater or reduced effectiveness, for example, having at least 25%, at least 50%, at least 75%, at least 100%, at least 150%, at least 300%, at least 500%, or at least 5000% of activation activity per molecule as compared to a naturally occurring or recombinant form of laminin) shall be considered “laminin”, provided that it can be used in lieu of the laminin in a defined cell culture media for growing and maintaining primate primordial stem cells in a substantially undifferentiated state. Matrigel™ is a soluble preparation from Engelbreth-Holm-Swarm tumor cells that gels at room temperature to form a reconstituted basement membrane. Other extracellular matrix components include fibronectin, collagen, and gelatin. In addition, one or more substances produced by the feeder cells, or contained in an extracellular matrix produced by a primate feeder cell line, can be identified and used to make a substrate that obviates the need for feeder cells. Alternatively, these components can be prepared in soluble form so as to allow the growth and maintenance of undifferentiated of stem cells in suspension culture. Thus, this invention contemplates adding extracellular matrix to the fluid phase of a culture at the time of passaging the cells or as part of a regular feeding, as well as preparing the substrate prior to addition of the fluid components of the culture.

Any suitable culture vessel can be adapted to culture stem cells (e.g., primate primordial stem cells) in accordance with the invention. For example, vessels having a substrate suitable for matrix attachment include tissue culture plates (including multi-well plates), pre-coated (e.g., gelatin-pre-coated) plates, T-flasks, roller bottles, gas permeable containers, and bioreactors. To increase efficiency and cell density, vessels (e.g., stirred tanks) that employ suspended particles (e.g., plastic beads or other microcarriers) that can serve as a substrate for attachment of feeder cells or an extracellular matrix can be employed. In other embodiments, undifferentiated stem cells can be cultured in suspension by providing the matrix components in soluble form. As will be appreciated, fresh medium can be introduced into any of these vessels by batch exchange (replacement of spent medium with fresh medium), fed-batch processes (i.e., fresh medium is added without removal of spent medium), or ongoing exchange in which a proportion of the medium is replaced with fresh medium on a continuous or periodic basis.

Genetic Engineering

General methods in molecular genetics and genetic engineering are described in the current editions of “Molecular Cloning: A Laboratory Manual” (Sambrook, et al., Cold Spring Harbor); Gene Transfer Vectors for Mammalian Cells (Miller & Calos eds.); and “Current Protocols in Molecular Biology” (Ausubel, et al. eds., Wiley & Sons). Cell biology, protein chemistry, and antibody techniques can be found in “Current Protocols in Protein Science” (Colligan, et al. eds., Wiley & Sons); “Current Protocols in Cell Biology” (Bonifacino, et al., Wiley & Sons) and “Current Protocols in Immunology” (Colligan et al. eds., Wiley & Sons.). Reagents, cloning vectors, and kits for genetic manipulation referred to in this disclosure are available from commercial vendors such as BioRad, Stratagene, Invitrogen, ClonTech, and Sigma-Aldrich Co.

Cell Culture Methods

The present invention is directed to compositions comprising conditioned hESC cell culture medium, or an extract thereof, generated using cell cultures and an appropriate carrier. The invention is also directed to methods for preparing such compositions. In certain embodiments, the culture comprises mammalian cells, preferably human cells, such as, for example, dermal fibroblasts, keratinocytes, epithelial cells, chondrocytes, smooth muscle cells, and myocytes. In certain embodiments the appropriate carrier is a pharmaceutically-acceptable carrier, a cosmetically-acceptable carrier. In certain embodiments, the conditioned cell culture media is generated using pre-conditioned media that is serum-free or animal product-free.

Human ESCs, derived from the inner cell mass, have the capacity for long-term undifferentiated growth in culture, as well as the theoretical potential for differentiation into any cell type in the human body. These properties offer hESCs as a potential source for transplantation therapies and as a model system for studying mechanisms underlying mammalian development. Human ESCs can be grown in a 6- or 12-well plate to maturation (day 6 or 7 after seeding) before being fixed and visualized under a phase contrast microscope. Cellular immunofluorescence can be used to assess the state of differentiation of hESCs. For example, hESCs are grown to maturation (day 6 or 7 after seeding) in a 12- or 24-well plate with a round cover slide in the bottom of each well. The cells are then fixed with 4% paraformaldehyde and blocked in PBS buffer containing 0.2% Triton X-100 and 2% BSA. Next, the cells were incubated with a primary antibody (Oct-4, SSEA-1, SSEA-3, SSEA-4, Tra-1-60, Tra-1-81, alkaline phosphatase, Myc, Map-2, Nkx2.5, bFGF (Santa Cruz Biotechnology, Inc.; Santa Cruz, Calif., world wide web: scbt.com) nestin, tyrosine hydroxylase (Chemicon International, Temecula, Calif., world wide web: chemicon.com), beta-tubulin (Sigma), p300, Tip60, or acetylated H4 (K5, 8, 12, 16) (Upstate Biotechnology, Lake Placid, N.Y., world wide web: upstate.com) in wash buffer (0.1% Triton X-100 in PBS) at 4° C. overnight, and then with secondary antibody (Molecular Probe; Eugene, Oreg.) in wash buffer at room temperature for 45 minutes. After further staining with DAPI, cells are mounted onto a microscope slide and visualized under an immunofluorescence and deconvolution microscope. The state of differentiation of HESC is further assessed by generating (via lentiviral-mediated transduction) hESCs carrying a reporter gene (enhanced green fluorescence protein (EGFP)) under control of the Oct-4 promoter. Using these transfected hESCs (carrying Oct-4 driven EGFP), the undifferentiated state of hESCs can be visualized by green fluorescence (indicating Oct-4 expression).

Examples of NIH-approved human ESC lines are lines H1 and H9, which can be obtained from the Wicell Research Institute (Madison, Wis.). Each cell line was originally maintained on mitomycin C-inactivated MEF (Specialty Media, Inc., Phillipsburg, N.J.) in media consisting of 80% DMEM/F-12 or KO-DMEM, 20% Knockout Serum Replacement, 2 mM L-alanyl-L-glutamine (GlutaMax) or L-glutamine, 1×MEM nonessential amino acids, 100 μM β-mercaptoethanol (all from Invitrogen, Carlsbad, Calif.), and 4 ng/mL bFGF (PeproTech Inc., Rocky Hill, N.J.). Cells were originally passaged once a week by treatment with dispase according to the instructions provided with the cell lines. Human ESCs on human feeder layers or on Matrigel-(Becton Dickinson, Bedford, Mass.) coated plates (see method of coating below) are maintained in DMEM/F-12 or KO-DMEM (80%), knockout Serum Replacement (20%), L-alanyl-L-glutamine or L-glutamine (2 mM), MEM nonessential amino acids (1.times.), P-Mercaptoethanol (100 μM), bFGF (20 ng/ml), and insulin (4 μg/ml). Human recombinant insulin was from Sigma (St. Louis, Mo.).

Any human ESC line may be used in the practice of the invention. Human ESC may be grown by any well known method and using any well known materials. Methods for obtaining, characterizing, and culturing human ESC's are described, for example, in Hoffman and Carpenter, Nature Biotech. 23:699-708, 2005 (review); Yao et al., Proc. Nat. Acad. Sci. USA 103:6907-6912, 2006; Bhattacharya et al., Blood 103:2956-2964, 2004; Cowan et al., “Derivation of Embryonic Stem-Cell Lines from Human Blastocysts, New England J. Med. 350(13), 2004; and Richards et al., Nature Biotech. 20:933-936, 2002. Methods for culturing human ESC's are provided Cell culture methods are also described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts useful include Creating a High Performance Culture (Aroselli, Hu. Res. Dev. Pr. 1996) and Limits to Growth (D. H. Meadows et al., Universe Publ. 1974). Tissue culture supplies and reagents are available from commercial vendors such as Invitrogen, Nalgene-Nunc International, Sigma Chemical Co., Chemicon International, and ICN Biomedicals.

Description of the growth of human ESC's on fibroblast feeder cells and in defined media are provided below as two examples of the many methods that can be used for growing human ESC's in order to provide conditioned medium for the practice of the present invention.

Growth of hESC on Fibroblast Feeder Cells. The following protocol is provided for illustrative purposes only; any well known method for growing hESCs can be used.

Two NIH-approved human ES cell lines (H9 and H14), were used with equal success to generate homogeneous cultures of human neural precursors. hESCs were grown with MEF feeders in knockout DMEM-20% serum replacement and 8 ng/ml bFGF. hESCs were collected by collagenase-IV treatment five days after plating. Cell clusters were allowed to settle and the supernatant containing floating cells was discarded. The hESC clusters were triturated to dislodge the loosely attached cells and to reduce the cluster size to ˜50-100 cells and washed twice with PBS. Small clusters (10-100 cells) of hESCs were grown in polypropylene dishes (Ted Pella) in Suspension Medium (1:1 ratio of DMEM/F12:neurobasal medium with N2+ B27 supplements, 20 ng/ml insulin 20 ng/ml bFGF, 20 ng/ml and EGF). The spheres were grown for six days, with a change in medium every alternate day. Spheres were collected, gently triturated and plated on ornithin coated (5 ng/ml, Sigma) plates (Corning) in the hNPC Expansion Medium (DMEM/F12, 10% BIT 9500, 20 ng/ml bFGF, 20 ng/ml EGF, 5 μg/ml fibronectin, 2 μg/ml heparin) for another six days to obtain hES-NPC monolayers. The clusters attached and appeared to be entirely composed of rosettes, with bipolar/triangular cells radiating outward. After 6 days in the expansion medium, the hESC-NPCs were replated in the same medium, to form monolayer cultures of hESC-NPCs.

In order to produce a fibroblast monolayer cell culture as feeder cells for growth of hESC, normal human dermal fibroblasts, isolated from a human foreskin, may be cultured in a 150 cm² tissue culture flasks (Corning, Corning, N.Y.) in monolayer culture using pre-conditioned cell culture media (in this example, high-glucose Dulbecco's Modified Eagle's Media (DMEM; GibcoBRL, Grand Island, N.Y.) supplemented with 10% bovine calf serum (Hyclone Laboratories, Logan, Utah), nonessential amino acids (GibcoBRL), and 100 U/ml penecillin-streptomycin-250 ng/ml amphoterecin B (GibcoBRL) (“DMEM 1”) in a 37° C., 5% CO₂ incubator. Monolayer cultures are fed twice weekly with fresh pre-conditioned media and passaged weekly using a 1 to 10 split, as described. See generally, Pinney et al., J. Cell. Physio. 183:74-82 (2000). The dermal fibroblasts may also be expanded in roller bottles with DMEM 1. The conditioned media from these monolayer cultures is collected and saved for future use.

While fibroblast cells have been used for illustrative purposes in this example, the skilled artisan will understand that many other types of cells, for example, but not limited to, other epithelial cell types, endothelial cells, smooth muscle cells, myocytes, keratinocytes, chondrocytes, and the like, may be grown in monolayer culture and in three-dimensional culture.

Human dermal fibroblasts can be seeded onto a variety of three-dimensional frameworks or suspended in a collagen matrix, using conventional technology. For example, cells can be seeded onto a bioabsorbable polyglactin mesh framework, such as Vicryl™ a substance commonly used for suture material that is composed of biodegradable mesh fibers of polyglactin 910 (a copolymer of 90:10 polyglycolic acid to polylactic acid) or a three-dimensional lactate/glycolate polymer framework.

Fibroblasts are cultured for approximately two weeks on a three-dimensional lactate/glycolate copolymer framework in antibiotic-free, high-glucose DMEM supplemented with 10% calf serum, 2 mM L-glutamine, non-essential amino acids, and 50 μg/ml ascorbate (J. T. Baker) (“DMEM 2”). In the presence of DMEM 2 and under conditions appropriate for cell growth, the fibroblasts proliferate to fill the interstices of the framework. The cells secrete collagen and other extracellular matrix components, growth factors, and cytokines, among other things, and create a three-dimensional human tissue, such as Dermagraft™, a tissue-engineered product developed for inter alia the treatment of diabetic foot ulcers (Advanced Tissue Sciences, La Jolla, Calif.); see Naughton, Dermal Equivalents, pp. 891-902, in Principles of Tissue Engineering, 2d ed., Lanza et al., eds., Academic Press, 2000.

The cultures are fed every 3-4 days with pre-conditioned DMEM 2 and the conditioned media was collected, starting after day 10, and either tested immediately or frozen at −20° C. for future testing. To quantitate the concentration of various growth factors and cytokines in one preparation of conditioned media, immunoassays are performed using the appropriate commercially available human growth factor ELISA kits (Quantikine™ Immunoassays, R & D Systems, Minneapolis, Minn.). Pre-conditioned DMEM 2 is assayed in parallel as a negative (background) control. Although the assays are identified as species specific, certain lots of bovine serum showed low levels of cross-reactivity in the TGFβ ELISA.

Depending on the cell type being cultured, many other types of cell culture media may be used. Exemplary cell culture media include Minimum Essential Medium Eagle (MEM), Keratinocyte Medium, Melanocyte Medium, Hepaotcyte Medium, Amniocyte Medium, Bone Marrow Medium, Basal Medium Eagle (BME), BGJb Medium (Fitton-Jackson Modification), Iscove's Modified Dulbecco's Medium (IMDM), L-15 Medium (Liebovitz), McCoy's 5A Modified Medium, MCDB Medium, Medium 199, Ham's F-10 Medium, Ham's F-12 Medium, RPMI-1640, Waymouth Medium, and the like; commercially available from, among others, Sigma-Aldrich, Life Technologies-GibcoBRL, or BioWhittaker.

An alternative three-dimensional fibroblast culture may be produced as follows. Passage 8 human dermal fibroblasts are seeded into conventional 1750 cm² corrugated roller bottles (Nalge or Nunc) containing a sterile nylon mesh scaffold (Industrial Fabrics) sitting on or near the inner surface of the roller bottle. The passage 8 fibroblasts are seeded at a density of approximately 4-6.times.10.sup.7 cells per roller bottle and cultured in antibiotic-free pre-conditioned media (DMEM (# 078-0521-189, Life Technologies-Gibco), supplemented with 2 mM L-glutamine (Life Technologies), non-essential amino acids (Life Technologies), 56 mg/l L-ascorbic acid (J. T. Baker), and 10% calf serum (HyClone Laboratories)). The roller bottles are incubated at 37° C. in a roller apparatus. The medium in the roller bottles is replaced daily or every other day using the pre-conditioned media described above and the conditioned cell culture media is collected. The VEGF level in the conditioned media is quantitated by ELISA, using the Quantikine human VEGF assay (R & D Systems, Minneapolis, Minn.) according to the manufacturer's instructions.

The person of skill in the art will understand that although roller bottles are described in this example, any number of bioreactors may be employed with appropriate modifications to the described conditions. The skilled artisan will also understand that any number of methods of processing the conditioned media, for example, but not limited to, chromatography, HPLC, phase separation, spray drying, evaporation, lyophilization, and the like, using methods known in the art.

Conditioned Serum-Free or Non-Human Animal Product-Free Media; Culturing Primate Stem Cells in a Defined Media

In certain embodiments of the invention, the conditioned media is generated using pre-conditioned media that is serum-free or animal product-free. Serum-free and animal product-free (sometimes referred to as protein-free) media is commercially available from, among other vendors, LifeTechnologies-GibcoBRL, Rockville, Md.; Sigma-Aldrich, Saint Louis, Mo.; or BioWhittaker, Walkersville, Md.). Exemplary serum-free media include: UltraCULTURE™, UltraDOMA™ and UltraCHO™, from BioWhittaker; Serum-free Hybridoma Medium, CHO Serum-free Medium, and MDCK Serum-free Medium, from Sigma-Aldrich; and Keratinocyte-SFM (KSFM), AIM V™ Media, StemPro™-34 SFM, Human Endothelial-SFM, Macrophage-SFM, and HepatoZYME-SFM from Life Technologies. Exemplary protein-free media include: UltraDOMA-PFT™ from BioWhittaker; Animal Component-free Hybridoma Medium, Serum-free and Protein-free Hybridoma Medium Hybri-Max™, CHO Protein-free Medium, Chemically-defined CHO Medium, and MDCK Protein-free Medium from Sigma-Aldrich; and Defined Keratinocyte-SFM from Life Technologies. The skilled artisan will appreciate that the use of serum-free media for mammalian cell culture is well established, and is described in, among other places, Cold Spring Harbor Conferences on Cell Proliferation, Vol. 9, Sato et al., eds., (1982) Cold Spring Harbor Laboratory, Cold Spring Harbor, N.Y.; Barnes et al., Anal. Biochem. 102, 255 (1980); BioWhittaker 1999/2000 catalog, pp. 42-51; Barnes, Serum-Free Animal Cell Culture, BioTechniques 5(6):534-42; and Freshney, Culture of Animal Cells, 3d ed., Wiley-Liss, New York, N.Y., 1994.

The media can be essentially serum-free, and does not require the use of a feeder cell layer or conditioned medium from separate cultures of feeder cells, although one can initially culture the stem cells in a growth environment that includes allogeneic feeder cells (or conditioned medium from such cells) prior to transferring the cells to fresh, feeder-free cultures for serial passaging (e.g., 1-50 or more passages). A defined cell culture medium thus can be used for growing and maintaining stem cells, including primate-derived stem cells, particularly primate primordial stem cells, in a substantially undifferentiated state. For example, such a culture medium solution can be isotonic, or have a low osmotic pressure. A basal medium that is effective to support the growth of, for example, primate-derived primordial stem cells can be supplemented with an amount of each of bFGF, insulin, and ascorbic acid necessary to support substantially undifferentiated growth of the stem cells. Such a medium may also include, without limitation, non-essential amino acids, an anti-oxidant, a reducing agent, growth factors, and a pyruvate salt. The base media may, for example be Dulbecco's Modified Eagle Medium (DMEM), DMEM/F-12, or KO-DMEM, each supplemented with L-glutamine or GlutaMAX™-I (provided as the dipeptide L-alanyl-L-glutamine (Invitrogen) at a final concentration of 2 mM), non-essential amino acids (1%), and 100 μM β-mercaptoethanol. A medium is preferably sterilized (e.g., by filtration) prior to addition to a cell culture.

U.S. Patent Application 60/435,991 (published as 20070010011) describes a representative example of a basal medium based on DMEM that can be used in practicing the invention. Other basal media useful in mammalian cell culture include, without limitation, Basal Media Eagle (BME), Glasgow Minimum Essential Media, Iscove's Modified Dulbecco's Media, Minimum Essential Media (MEM), Modified Eagle Medium (MEM), Opti-MEM I Reduced Serum Media, RPMI Media 1640, Waymouth's MB 752/1 Media, Williams Media E, Medium NCTC-109, neuroplasma medium, BGJb Medium, Brinster's BMOC-3 Medium, CMRL Medium, C02-Independent Medium, Leibovitz's L-15 Media, McCoy's 5A Media (modified), and MCDB 131 Medium.

Exogenous growth factors may also be added to a medium according to the invention to assist in the maintenance of cultures of stem cells (e.g., primate primordial stem cells) in a substantially undifferentiated state. Such factors and their effective concentrations can be identified as described elsewhere herein or using techniques known to those of skill in the art of culturing cells. Representative examples of growth factors useful in this regard include bFGF, insulin, acidic FGF (aFGF), epidermal growth factor (EGF), insulin-like growth factor I (IGF-I), IGF-II, platelet-derived growth factor (PDGF), and vascular endothelial growth factor (VEGF), activin-A, bone morphogenic proteins (BMPs), forskolin, glucocorticords (e.g., dexamethasone), transferring, and albumins.

Useful reducing agents include β-mercaptoethanol. In a preferred embodiment, the β-mercaptoethanol is present in a concentration of about 0.1 mM. Other reducing agents such as monothioglycerol or dithiothreitol (DTT), alone or in combination, can be used to similar effect. Still other equivalent substances will be familiar to those of skill in the cell culturing arts.

Pyruvate salts may also be included in a medium according to the invention. Pyruvate salts include sodium pyruvate or another pyruvate salt effective maintaining and/or enhancing primate primordial stem cell growth in a substantially undifferentiated state such as, for example, potassium pyruvate. In preferred embodiments, the pyruvate salt is added to a concentration of about 0.1 mM.

Other compounds suitable for supplementing a culture medium of the invention include nucleosides (e.g., adenosine, cytidine, guanosine, uridine, and thymidine) and nucleotides. Nucleosides and/or nucleotides can be included in a variety of concentrations, preferably ranging from about 0.1 μM (micromolar) to about 50 μM.

In preferred embodiments, a medium's endotoxicity, as measured in endotoxin units per milliliter (“eu/mI”), will be less than about 0.1 eu, and, in more preferred embodiments, will be less than about 0.05 eu/mI. In particularly preferred embodiments, the endotoxicity of the base medium will be less than about 0.03 eu/ml. Methods for measuring endotoxicity are known in the art. For example, a preferred method is described in the “Guideline on Validation of the Limulus Amebocyte Lysate Test as an End-product Endotoxin Test for Human and Animal Parental Drugs, Biological Products and Medical Devices,” published by the U.S. Department of Health and Human Services, FDA, December 1987.

As will be appreciated, it is desirable to replace spent culture medium with fresh culture medium either continually, or at periodic intervals, preferably every 1 to 3 days. One advantage of using fresh medium is the ability to adjust conditions so that the cells expand more uniformly and rapidly than they do when cultured on feeder cells according to conventional techniques, or in conditioned medium.

Populations of stem cells (such as primate primordial stem cells) can be obtained that are 4-,10-, 20-, 50-, 100-, 1000-, or more fold expanded when compared to the previous starting cell population. Under suitable conditions, cells in the expanded population will be 50%, 70%, or more in the undifferentiated state, as compared to the stem cells used to initiate the culture. The degree of expansion per passage can be calculated by dividing the approximate number of cells harvested at the end of the culture by the approximate number of cells originally seeded into the culture. Where geometry of the growth environment is limiting or for other reasons, the cells may optionally be passaged into a similar growth environment for further expansion. The total expansion is the product of all the expansions in each of the passages. Of course, it is not necessary to retain all the expanded cells on each passage. For example, if the cells expand two-fold in each culture, but only about 50% of the cells are retained on each passage, then approximately the same number of cells will be carried forward. But after four cultures, the cells are said to have undergone an expansion of 16-fold. Cells that are not passaged forward may be retained, if desired, in which event they may be frozen and stored, preferably in liquid nitrogen or at −140° C.

Of course, culture conditions inappropriate for stem cells such as primate primordial stem cells will cause the cells to differentiate promptly, although it will be appreciated that marginally beneficial conditions may allow the stem cells to go through a few passages while still retaining a proportion of undifferentiated cells. In order to test whether conditions are adequate for indefinite culture of stem cells (e.g., primate primordial stem cells) in a substantially undifferentiated state, it is recommended that the cells be expanded in a preferable range of about 4- to about 10-fold every passage. A higher degree of expansion and/or a higher number of passages (e.g., at least 11 passages) provides a more rigorous test. An effective test for whether a cell population is substantially undifferentiated is the demonstration that the cells express cell surface markers indicative of an undifferentiated state.

Human dermal fibroblast cultures grown in serum-containing pre-conditioned DMEM media can be adapted to growth in pre-conditioned UltraCULTURE. serum-free media using conventional technology. See, e.g., BioWhittaker 1999/2000 Catalog at pages 42-45. UltraCULTURE™. (BioWhittaker Cat. No. 12-725F) media is supplemented with L-glutamine (Cat. No. 17-605) according to the manufacturer's instructions (pre-conditioned UltraCULTURE™ serum-free media).

Monolayer cultures of human dermal fibroblasts are prepared using pre-conditioned DMEM cell culture media (high-glucose Dulbecco's Modified Eagle's Media (DMEM; GibcoBRL, Grand Island, N.Y.) supplemented with 10% bovine calf serum (Hyclone Laboratories, Logan, Utah), nonessential amino acids (GibcoBRL), and 100 U/ml penicillin-streptomycin-250 ng/ml amphoterecin B (GibcoBRL). The cells are passaged, and split 1:2 using pre-conditioned UltraCULTURE™ serum-free media as the diluent. The cells are plated and incubated in a 37° C., 5% CO₂ incubator until maximum cell density is achieved, feeding with pre-conditioned UltraCULTURE™ serum-free media as necessary.

If the cells do not show at least 85% viability, they are passaged at a 1:2 ratio using pre-conditioned UltraCULTURE™. serum-free media supplemented with 0.5% bovine calf serum (HyClone Laboratories) for one passage. For each successive passage the amount of calf serum is decreased by 0.1% so that after five passages, the pre-conditioned UltraCULTURE™ serum-free media contains no serum. At this point the fibroblasts can be propagated in three-dimensional culture, with the exception that the cells are maintained in pre-conditioned UltraCULTURE™ serum-free media, supplemented with ascorbic acid as appropriate. Conditioned serum-free media is collected at suitable intervals.

If the fibroblast monolayer culture does not successfully adapt to growth in pre-conditioned UltraCULTURE™ serum-free media, an alternate weaning process is used. Cells are passaged as described, centrifuged for 5 minutes at 350.times.g and resuspended in pre-conditioned UltraCULTURE™ serum-free media containing 5% bovine calf serum (Hyclone), split 1:2 and replated. At the next passage, the cell pellet is resuspended in pre-conditioned UltraCULTURE™ serum-free media containing 2% calf serum, split and plated, as described. On the next five passages, the pellet is resuspended and plated in pre-conditioned UltraCULTURE™ serum-free media containing 2%, then 1%, then 0.5%, then 0.1%, and finally 0% calf serum. At this point the fibroblasts can be propagated in three-dimensional culture, as described in Examples 2 or 3, with the exception that the cells are maintained in pre-conditioned UltraCULTURE™ serum-free media. Conditioned media is collected as appropriate.

The use of UltraCULTURE™ serum-free media is described because, among other things, it is a DMEM-based medium and has been shown to support the growth of a number of human cell lines, including the HuS-1* AT skin cell line. (BioWhittaker). The skilled artisan will appreciate, however, that a number of serum-free and animal product-free media are also reasonably likely to support the growth of various human cells and that such media can be routinely evaluated without undue experimentation.

Growth of hESC on Defined Medium. The following is a description of the use of a defined medium for growth of hESC as one example.

Initially, the hESC lines are maintained on growth-arrested MEFs. The undifferentiated hESCs formed tightly packed colonies with small compact cells of high nucleus-to-cytoplasm ratio. The hESC colonies then expand by anchorage to surrounding feeder cells and by loosely attaching to the underlying tissue culture plate. Cells are initially passaged by treatment with dispase once a week. However, if dispase treatment does not efficiently separate hESCs from surrounding MEF cells or effectively dissociate hESC colonies during passaging, additional mechanical dissection steps may be required to detach and break hESC colonies down to smaller pieces. Trypsin treatment is not an acceptable alternative in those culture conditions, because treatment sufficient to dissociate the cells can be lethal to undifferentiated hESCs on feeder layers; and the HESC colonies that survive can have an unacceptably higher rate of spontaneous differentiation than the parent colonies. As a result, non-enzymatic dissection process can be used that produces more uniformly undifferentiated HESC colonies than the enzymatic methods. In this procedure, colonies estimated as having more than 80% morphologically undifferentiated cells are selected to be split. The selected hESC colonies are separated from the surrounding feeder cells, sliced into pieces, and detached from the tissue culture plate with a sterile plastic pipette tip. Then, the dissected HESC colony pieces are transferred to a fresh feeder layer and allowed to attach overnight. Culture medium is replaced every other day. The hESCs are passaged by this procedure every seven days at a split ratio of 1:8 to 1:4. This procedure not only is less time-consuming, but also can result in higher plating efficiencies and more uniformly undifferentiated HESC colonies than the enzymatic methods.

In order to establish a culture system that is free of non-human animal products, the human foreskin fibroblast (HFF) cell line Hs27, as one example, can be used as a feeder layer. The human foreskin fibroblast (HFF) cell line, Hs27 (American Type Culture Collection [ATCC]; Manassas, Va.) is expanded to create a master bank of frozen cells. The HFFs are plated in gelatin pre-coated 60 mm plates or 6-well plates at a density of 1.7.times.10⁴/cm² and inactivated by irradiating at 50 Gy using a ¹³⁷Cs gamma-irradiator before being used as feeder cells. Undifferentiated hESCs, although originally maintained on MEFs, were transferred to plates of HFFs that had been mitotically inactivated by gamma irradiation. In the first attempts to transfer the hESCs to the human feeder layers, far more differentiated cells compared to those grown on MEFs may be observed. When dealing with hESCs, the undifferentiated state is assessed by three criteria: (a) distinctive and defining stage-specific morphology and size; (b) the expression of immunomarkers associated with pluripotency; and (c) the absence of immunomarkers associated with lineage commitment. The hESC colonies maintained on HFFs display a more irregular morphology, and are more elliptical and less round than those grown on MEFs. Human ESC colonies co-cultured with HFFs are considerably smaller than those grown on MEFs, suggesting that some of the factors produced by MEFs that support undifferenfiated HESC growth are missing or insufficient in the HFF culture system. Immunostaining for the undifferentiated HESC markers Oct-4, SSEA4, Tra-1-60, and Tra-1-81 indicated that the HESC colonies on HFFs contained mixed patches of undifferentiated (<30%) and differentiated cells, often separated by distinct borders.

By increasing the bFGF concentration in the HESC medium to 20 ng/ml (from 4 ng/ml), the HESC colonies grown on the HFFs display the more round and undifferentiated morphological characteristics of HESC colonies grown on MEFs. These hESC colonies are also significantly larger, suggesting that bFGF promoted undifferentiated growth of hESCs on feeder layers. In addition to bFGF (20 ng/ml), the medium used to obtain these results contains DMEM/F-12 or KO-DMEM (80%), knockout Serum Replacement (20%), L-alanyl-L-glutamine or L-glutamine (2 mM), MEM nonessential amino acids (1×), and β-mercaptoethanol (100 μM). In this media, less than 80% undifferentiated HESC colonies are observed on HFF feeders on every passage. Using this system, undifferentiated hESCs on HFF feeder layers have been maintained for over 12 months ( more than 50 passages), thereby exhibiting sustained long-term undifferentiated growth as assessed both by morphological and immunological criteria. Specifically, hESCs maintained on HFFs displayed uniform undifferentiated morphology as well as high expression levels of Oct-4, SSEA-4, Tra-1-60, and Tra-1-81, but not SSEA-1. Only cells at the edge of the colonies exhibited the classic signs of early differentiation: flat epithelial cell-like morphology; expression of the cell surface marker SSEA-3 and the neural/beta-cell precursor marker nestin. Cells that migrated beyond the edge of the colonies continued, as classically observed, to differentiate further into large elliptical cells that persisted in expressing nestin (suggestive of neuroectodermal commitment) and appropriately now downregulated SSEA-3 (FIG. 1a, B, red arrows).

Bovine FGF (bFGF) can be used to promote undifferentiated growth of hESCs on matrix proteins in the absence of feeder cells. Gelatin pre-coated plates are incubated with a commercially-available combination of laminin and collagen known as Matrigel (Growth factor reduced, Becton Dickinson) [diluted 1:30 in cold DMEM/F-12] at 4° C. overnight. Over 80% of HESC colonies maintained on laminin/collagen-coated, plates in each passage are highly compact and undifferentiated, as evidenced by their morphology and expression of Oct-4, SSEA4, Tra-1-60, and Tra-1-81 by day 7. The colonies on laminin/collagen have a more uniform morphology than those grown on HFFs, as indicated by the presence of an even narrower edge of SSEA-3-positive “transitional” (imminently-differentiating) cells. Undifferentiated HESC colonies can be maintained sustained long term on laminin/collagen-coated plates.

In media supplemented with bFGF at a concentration ranging from 10 to 50 ng/ml, hESCs display a growth rate comparable to that maintained in MEF-CM, while the optimal proportion of undifferentiated hESCs, comparable to MEF-CM, appear to be maintained at 20 ng/ml bFGF. At the proper concentration, bFGF may substitute for feeder cells or MEF-conditioned media. In addition to bFGF, insulin and ascorbic acid are also essential—perhaps in a collaborative manner—for maintaining substantial numbers of hESCs in a healthy undifferentiated state. Although albumin and transferrin are not crucial components for sustaining the undifferentiated growth of hESCs, they may contribute to survival or maintenance of a normal colony shape.

Similar to hESCs maintained on laminin/collagen-coated plates, more than 80% of HESC colonies remain undifferentiated on surfaces coated with laminin alone, as indicated by their classic undifferentiated morphology and their expression of Oct-4. In contrast, the majority of the HESC colonies (more than 70%) maintained on human fibronectin, human collagen IV, or, as a control, gelatin-coated plates, display a more differentiated morphology upon the first passage, leaving only a minority (less than 30%) of small colonies bearing a compact, undifferentiated morphology. Interestingly, the colonies of undifferentiated cells maintained under the feeder-free conditions (on either laminin or laminin/collagen-coated plates) appeared to be associated with a monolayer of hESC-derived fibroblastic cells that expressed nestin and vimentin. These cells may spontaneously act as “auto feeder layers” for the very same undifferentiated HESC colonies from which they were derived, preventing them from differentiating, rendering the system “self-contained”, “self-supporting”, and precluding the need for exogenous “biologics”—including human-derived components.

To demonstrate the self-renewal of undifferentiated hESCs maintained under the above-described defined biologics-free culture conditions, hESCs are treated with trypsin, dissociated into a single cell suspension, and then cultivated under the defined conditions. Undifferentiated mature-sized single-cell-derived hESC colonies begin to appear after 4-7 days in vitro. A 12.6±3.8% cloning efficiency of hESCs cultured under the defined conditions is observed. This observation contrasts starkly with the extremely poor cloning efficiency that has been reported previously using culture conditions employing feeder cells or conditioned media. In fact, complete cell death has been observed when single undifferentiated cells dissociated by trypsin treatment were passaged onto exogenous feeder cells or in conditioned media (particularly for hESCs that have never been exposed to trypsin digestion, e.g., HES-25). However, undifferentiated hESCs display unexpectedly high passaging efficiency with trypsin treatment under the defined biologics-free culture conditions. One explanation is that the dissociated single cells seeded highly efficiently on a substrate containing laminin in the defined HESC media. In addition, the colonies of undifferentiated cells appeared to be associated with a monolayer of hESC-derived fibroblastic cells that expressed vimentin. These differentiated cells may spontaneously act as “auto feeder layers” for the very same undifferentiated HESC colonies from which they were derived, preventing them from differentiating. Stated another way, the system appears become “self-contained” or “self-supporting” since pluripotent hESCs will inevitably include, among its many products of differentiation, those lineages that have heretofore been supplied exogenously as “foreign” human feeder cells. The system allows these hESCs to produce their own support (“feeder”) cells.

In summary, a defined serum-free, conditioned medium-free medium can be used for the long-term cultivation of undifferentiated hESCs on not only human feeder layers but also under feeder-free conditions. Using this defined culture system hESCs can be expanded efficiently and stably following trypsin-mediated dissociation.

Filtering and Concentrating Conditioned Media. The conditioned media is pre-filtered to remove large particulate, such as cell debris, for example, using a 3M.TM.522 High Performance Liquid Filter Bag (Southcoast) with a 2.5 micron rating to produce “filtered media” (also referred to as 1× conditioned media). For certain applications the filtered media is concentrated in a cross flow hollow fiber uiltrafiltration cartridge (Model #UFP-10-C-55A, A/G Technology Corp., Needham, Mass.) at a flow rate of 25 liters per minute, according to the manufacturer's Operating Guide. The “nutrient solution,” (also referred to as 10× conditioned media) concentrated to approximately three to fifteen times the initial concentration, is collected.

The 1×- and 10×-conditioned media is used by formulators for preparing compositions comprising cosmetic, cosmeceutical, or pharmaceutical formulations with cosmetically-acceptable, cosmeceutically-acceptable or pharmaceutically-acceptable carriers. The skilled artisan will appreciate that cosmetically-acceptable carriers, cosmeceutically-acceptable carriers and pharmaceutically-acceptable carriers may be the same or different, depending on the intended application of the composition.

Cosmetics, Cosmaceuticals, and Related Methods of Use

The term cosmeceutical refers to a formulation or composition comprising at least one biologically active ingredient that has an effect on the user of the product and at least one cosmeceutically-acceptable carrier. Cosmeceuticals may be viewed as cosmetics that, in certain applications and under appropriate conditions, may provide medicinal or drug-like benefits. In certain applications, for example, cosmeceuticals may affect the underlying structure of the skin, decrease wrinkle depth, or reverse or ameliorate the effect of photooxidation or aging on the skin. Cosmeceuticals may be particularly useful as skin care products, hair care products, and sun care products. In certain embodiments, cosmeceutical compositions comprise delivery systems including at least one of liposomes, cyclodextrins, polymer systems, or hyaluronic acid or related compounds. Cosmeceutical compositions comprise cosmeceutically-acceptable carriers. The skilled artisan will understand that a pharmaceutically-acceptable carrier or formulation that is suitable for topical applications will typically also be a cosmeceutically-acceptable carrier or formulation.

A topical cosmetic or cosmeceutical ointment, lotion, or gel composition typically contains an effective amount of conditioned media or extracts thereof and may comprise other active and inert ingredients as well in a cosmetically- or a cosmeceutically-acceptable carrier, such as a pharmaceutical cream base, an oil-in-water emulsion, a water-in-oil emulsion, a gel, or the like. Various cosmetic and cosmeceutical compositions for topical use include drops, tinctures, lotions, creams, salves, serums, solutions, and ointments containing conditioned media or extracts, and an appropriate carrier. The optimal percentage of the conditioned media or extract in each composition varies according to the composition's formulation and the therapeutic effect desired.

The skilled artisan in the formulation arts will understand that the inventive compositions may comprise any of a number of cosmetically-, cosmeceutically-, or pharmaceutically-acceptable formulations, depending on the type of product, the nature of the composition, the location of composition's use, the desired effect, and the like. While proprietary formulations are common in the formulation arts, formulators of ordinary skill will be able to determine or readily select appropriate formulations for specific applications without undue experimentation.

Discussions of cosmetic- and cosmeceutically-acceptable ingredients and formulations may be found in, among other places, FDA Cosmetics Handbook, U.S. Food and Drug Administration; Handbook of Cosmetic and Personal Care Additives, Ash and Ash, compilers, 1994, Chemical Publishing, New York, N.Y.; Bennett's Cosmetic Formulary, 1993, Chemical Publishing Co.; Harry's Cosmeticology, 7.sup.th ed., Wilkinson & Moore, 1982 and 8.sup.th ed., Rieger, 2000, Chemical Publishing; Cosmetic Bench Reference-2001, Allerud Publishing Corp.; CTFA Compendium of Cosmetic Ingredient Composition, Nikitakis and McEwen, eds., 1990, Cosmetic, Toiletry, and Fragrance Association, Washington, D.C., Surfactant Encyclopedia, 2.sup.nd revised edition, Rieger, 1996, Allured Publishing; The Chemistry and Manufacture of Cosmetics, 2.sup.nd ed., De Navarre, Van Nostrand, Princeton, N.J.; Encyclopedia of Common Natural Ingredients Used in Food, Drugs, and Cosmetics, Leung, 1996, John Wiley; A Consumer's Dictionary of Cosmetic Ingredients, 5.sup.th ed., Winter, 1999, Three Rivers Press, New York, N.Y.; Cosmeceuticals: Active Skin Treatment, 1998, Allured Publishing; Handbook of Cosmetic Science and Technology, Knowlton and Pearce, 1993, Elsevier Advanced Technology, Oxford, UK; Personal-Care Formulas, 1997, Allured Publishing; Beginning Cosmetic Chemistry, Scheuller and Romanowski, 1999, Allured Publishing; and Skin Permeation: Fundamentals and Application, Zatz, 1993, Allured Publishing. Discussions of pharmaceutically-acceptable ingredients and formulations may be found in, among other places, Remington's Pharmaceutical Sciences, 18.sup.th ed., Gennaro, ed., 1990, Mack Publishing.

The methods of the invention have wide applicability to cosmetic conditions. The mode of administration for cosmetic applications is typically topical, but administration and dosage regimens will vary depending on the cosmetic condition whose modulation is sought.

The present invention provides methods, compositions, and kits for cosmetic use with individuals. The term “individual” as used herein includes humans as well as other mammals. In some embodiments, the compositions, methods, and/or kits are used to provide a cosmetic treatment to an individual desiring and/or in need of cosmetic treatment (e.g., young children subject to bum or other scarring may not desire treatment but may nonetheless be in need of treatment). The term “treating” or “treatment” as used herein includes achieving a cosmetic benefit. By cosmetic benefit is meant any desired modulation of the cosmetic condition being treated. For example, in an individual with wrinkling, cosmetic benefit includes eradication or lessening of the appearance of wrinkling. Also, a cosmetic benefit is achieved with the eradication or amelioration of one or more of the psychological symptoms associated with the underlying condition such that an improvement is observed in the patient, notwithstanding the fact that the patient may still be affected by the cosmetic condition. For example, conditioned medium provides cosmetic benefit not only when a cosmetic defect is eradicated, but also when an improvement is observed in the individual with respect to the cosmetic defect and its attendant consequences, such as psychological consequences. In some cases, methods and compositions of the invention may be directed at achieving a prophylactic benefit. A “prophylactic,” or “preventive” effect includes prevention of a condition, retarding the progress of a condition (e.g., skin aging), or decreasing the likelihood of occurrence of a condition. As used herein, “treating” or “treatment” includes prophylaxis.

As used herein, the term “effective amount” encompasses an amount sufficient to effect beneficial or desired cosmetic results. An effective amount can be administered in one or more administrations. In terms of cosmetic treatment, an “effective amount” of conditioned medium of the invention is an amount that is sufficient to palliate, ameliorate, stabilize, reverse or slow the progression of a cosmetic condition, or to provide a desired effect such as cosmetic augmentation of a soft tissue. An “effective amount” may be of a conditioned medium used alone or in conjunction with one or more agents used to modulate a cosmetic condition.

The skin is subject to a number of cosmetic conditions that result in alterations of function and/or appearance that are considered undesirable, and whose manifestation can lead to psychological discomfort as well as, in some cases, physiological discomfort or harm. In some cases, although no defect is present, it is nonetheless desirable to the individual to augment or alter the skin in such a way as to produce a cosmetically pleasing effect.

Exemplary cosmetic conditions that may be modulated by the methods of the invention include, but are not limited to, skin aging, cosmetic defect, undesired pigmentation, and post-cosmetic procedure damage.

Skin aging includes chronological aging as well as photoaging, and may appear as wrinkling, lack of elasticity (e.g. sagging), uneven pigmentation, thinning of the skin and/or collagen so that veins and other underlying structures become more prominent, and the like. Skin aging is a major example of a skin condition that involves a decrease in cell proliferation and in cell function. As used herein, “skin aging” refers to alterations in the appearance and function of skin that occur with aging, such as wrinkling, loss of elasticity, sagging, uneven pigmentation (e.g., “age spots” or “liver spots”), and loss of underlying tissue mass. Such conditions may be accelerated and/or exacerbated by exposure to ultraviolet radiation (“photoaging”) and other environmental conditions. With age and/or exposure to UV radiation, the epidermis thins and the skin appendages atrophy. Hair becomes sparse and sebaceous secretions decrease, with consequent susceptibility to dryness, chapping, and fissuring. The dermis diminishes with loss of elastic and collagen fibers. Moreover, keratinocyte proliferation (which is indicative of skin thickness and skin proliferative capacity) decreases with age.

An increase in keratinocyte proliferation and collagen production is believed to counteract skin aging, i.e., wrinkles, thickness, elasticity and repair. According to the present invention, conditioned medium can be used cosmetically to counteract, at least for a time, the effects of aging on skin. A formulation containing conditioned medium may be applied topically in areas where it is desired to counteract skin aging.

Also included in the skin conditions that may be treated by the methods of the invention are cosmetic defects that, while not pathological or physiologically harmful, may nonetheless cause psychological distress, in some cases to the extreme. In these cases it is desirable to correct a particular feature or features causing distress or, alternatively, enhance a feature considered desirable. In addition to skin aging, such conditions include, e.g., striae gravidorum and striae distensiae (“stretch marks), atrophic scarring (e.g., acne scarring), wound (e.g., traumatic wounds, chronic wounds, or bum wounds) or surgical scarring, thickened and cracked skin (especially on the feet), and hair loss.

In the latter embodiments, the invention relates to the use of preparations that comprise conditioned medium to enhance hair growth. Cells from which the hair is produced grow in the bulb of the follicle. They are extruded in the form of fibers as the cells proliferate in the follicle. Hair “growth” refers to the formation and elongation of the hair fiber by the dividing cells. In some embodiments, the methods of the invention provide a means for altering the dynamics of the hair growth cycle to induce proliferation of hair follicle cells, particularly stem cells of the hair follicle. The subject compositions and method can be used to increase hair follicle size and the rate of hair growth in individuals, such as humans, e.g., by promoting proliferation of hair follicle stem cells. In one embodiment, the method comprises administering to the skin in the area in which hair growth is desired an amount of conditioned medium sufficient to increase hair follicle size and/or the rate of hair growth in the animal, e.g., human. Typically, the composition will be administered topically as a cream or lotion, and will be applied on a daily basis until hair growth is observed and for a time thereafter sufficient to maintain the desired amount of hair growth.

Undesired pigmentation includes pigmentation over an area of the body that is different than the pigmentation desired by the individual. Undesired pigmentation can be the result of, e.g., photoaging, reaction to inflammation, or reaction to trauma such as surgical or accidental skin breakage, and the like. Undesired pigmentation includes altered or undesired pigmentation over small areas such as freckles, as well as altered or undesired pigmentation over larger areas, such as, for example, uneven pigmentation or larger areas of undesired pigmentation.

Further cosmetic uses of the methods of the invention include tissue augmentation through, generally, topical application, such as for lip enhancement. By “augment” is meant to include giving the appearance of greater fullness, generally through an increase in the tissue of the skin or underlying tissue. Any suitable skin area may be selected for augmentation by the methods of the invention.

In addition, the methods of the invention may be employed to enhance and/or accelerate recovery from standard cosmetic procedures, which are in themselves damaging to skin and/or underlying tissues, and which may take undesirably lengthy periods of time for recovery and/or may produce suboptimal results. Such procedures include chemical peel, dermabrasion, laser resurfacing, ablative resurfacing, nonablative resurfacing, photodynamic therapy, noncoherent light phototherapy, breast lift, face lift, eyelid lift, forehead lift, neck lift, thigh lift, buttock lift, tummy tuck, and scar revision. As will be apparent to those of skill in the art, some of these procedures can require further skin firming (e.g., “lifting” procedures) while others are more extensively damaging to the surface of the skin and require assistance for healing in a timely and optimal fashion (e.g., chemical peel, dermabrasion, ablative and non-ablative skin resurfacing). In some embodiments, the methods of the invention provide a method for achieving firming and lifting of the eyelids; this may be done either in place of or in conjunction with a conventional eyelid lift procedure. The methods of the invention may be used in conjunction with both types of procedures to enhance and/or accelerate healing and recovery.

Conditioned mediums may be administered in any cosmetically acceptable carrier, as described in more detail below. In embodiments of methods of the invention, the concentration of conditioned medium used may be more than about 0.00001, 0.00005, 0.0001, 0.001, 0.01, 0.1, 1, 5, 10 or 20%. In some embodiments, the concentration of conditioned medium is more than about 0.00005%. The concentration of conditioned medium may be less than about 0.0001, 0.001, 0.001, 0.01, 0.1, 1, 5, 10, or 20% (all concentration percentages given herein are w/w unless otherwise indicated). In some embodiments, the conditioned medium is used at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations are contemplated. Skin coverage may also be described in terms of total μL of conditioned medium/cm² of skin; in these terms, a typical coverage per administration would be more than about 3, 6, 60, 600, 6000, 60,000, or 600,000 μL/cm² of skin; less than about 900,000, 600,000, 60,000, 6000, 600, 60, or 6 μL/cm² of skin; or about 6 μL/cm² to about 600 μL/cm² of skin; or about 60 μL/cm² of skin.

The methods of the invention typically utilize topical administration, which may be by any suitable means that brings the conditioned medium and, optionally, other cosmetic or dermatological agents, in contact with the surface of the skin, including application as a gel, lotion, cream, liposomal preparation or the like, with or without occlusion, or application as a plaster, patch, mask, glove, or similar device for extended contact with an affected area of skin. For modulation of cosmetic conditions or to produce a desired cosmetic effect, the frequency and duration of administration of a formulation comprising a Conditioned medium is dependent on factors including the nature of the formulation (e.g., concentration, presence or lack of other cosmetic or dermatological agents, vehicle type), the severity and extent of the condition, and in some cases the judgment of a skin care professional, e.g., a health care professional such as a dermatologist, or a cosmetologist.

Topical application may be more than about once, twice, three times, four times, five times, or six times per week, or more than once, twice, three times, four times, five times, or six times per day. Frequency of application may be less than about twice, three times, four times, five times, or six times per week, or less than about once, twice, three times, four times, five times, or six times per day. Some embodiments of the invention provide a method for cosmetic treatment of the skin of an individual by topical administration of an effective amount of a Conditioned medium. In some embodiments, the formulation is administered an average of about once per day; in some embodiments, the formulation is administered an average of about once or twice per day; in some embodiments, the formulation is administered an average of about once to three times per day; in some embodiments, the formulation is administered an average of more than about three times per day. In one embodiment, the formulation is administered an average of about twice per day, typically in the morning upon rising and in the evening before retiring. Topical administration may be without a covering. Alternatively, topical administration may include the use of a covering over the formulation, which may be occlusive or non-occlusive. For example, administration in the evening before retiring may include covering the administered area with an occlusive or non-occlusive covering, which may remain in place during sleep.

The duration of treatment generally will depend on the response of the skin to cosmetic treatment. Treatment may continue at the discretion of the individual being treated. In some cases, administration or application of a formulation containing conditioned medium may be more frequent at the beginning of treatment and less frequent as treatment continues and the condition is ameliorated or the desired effect is achieved. In some cases, treatment may continue indefinitely in order to maintain a condition in abeyance or in an improved state, to delay onset of a cosmetic condition (e.g., skin aging), or to slow the progression of a cosmetic condition. These modifications of frequency and duration are easily accomplished by the individual being treated.

Some embodiments of cosmetic treatment of skin employ topical administration of a lotion, in some embodiments a mixture of emulsifying lanolin alcohols, waxes, and oils (e.g., EUCERIN™) or a mixture of petrolatum or mineral oil, a quaternary ammonium compound, a fatty alcohol, and a fatty ester emollient (e.g., CUREL™, see U.S. Pat. No. 4,389,418), as described below, comprising conditioned medium. The lotion containing the conditioned medium is applied at a frequency of once to three times per day, in some embodiments once per day, until the desired result, e.g., reduction or elimination of wrinkling, sagging, and the like, is observed, followed by topical application once to three times per week, in some embodiments once per week, thereafter.

The skilled artisan will understand that the appropriate carriers of the inventive compositions typically may other ingredients typically found in the cosmetic and cosmeceutical fields: oils, waxes or other standard fatty substances, or conventional gelling agents and/or thickeners; emulsifiers; moisturizing agents; emollients; sunscreens; hydrophilic or lipophilic active agents, such as ceramides; agents for combating free radicals; bactericides; sequestering agents; preservatives; basifying or acidifying agents; fragrances; surfactants; fillers; natural products or extracts of natural product, such as aloe or green tea extract; vitamins; or coloring materials. The amounts of these various ingredients will vary depending on the use of the composition and the cosmetic or cosmeceutical effect desired.

If the conditioned medium is used in combination with another skin care method or composition, any suitable combination of the conditioned medium and the additional method or composition may be used. Thus, for example, if use of a conditioned medium is in combination with another cosmetic or dermatological agent, the two may be administered simultaneously, consecutively, in overlapping durations, in similar, the same, or different frequencies, etc. In some cases a composition will be used that contains a conditioned medium in combination with one or more other cosmetic or dermatological agents.

Other dermatological or cosmetic agents that may be used in methods of the invention are described in more detail below. Dosages, routes of administration, administration regimes, and the like for these agents are well-known in the art.

As the cosmetic compositions are for topical use, they need not be sterile; however, if sterility is desired it is readily accomplished by filtration through sterile filtration (0.22 micron) membranes, or by other art-accepted means.

If desired, further cosmetic or dermatological ingredients may be incorporated in the formulations. The nature of the other ingredient(s) will depend on the cosmetic condition to be modulated and/or cosmetic result desired. These are described more fully below.

The conditioned medium may be used neat (e.g., with an occlusive dressing so that the conditioned medium is dissolved or dispersed in perspiration at the site), but generally is prepared in a vehicle suitable for topical administration. Compositions of the invention include conditioned medium in a vehicle suitable for topical administration.

Numerous vehicles for topical application of cosmetic compositions are known in the art. See, e.g., Remington's Pharmaceutical Sciences, Gennaro, A R, ed., 20.sup.th edition, 2000: Williams and Wilkins PA, USA. All compositions usually employed for topically administering cosmetic compositions may be used, e.g., creams, lotions, gels, dressings, shampoos, tinctures, pastes, ointments, salves, powders, liquid or semi-liquid formulation, patches, liposomal preparations, and the like. Application of said compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used. The conditioned medium may optionally be dissolved or diluted in a small amount of an appropriate solvent, such as ethanol or DMSO, before dispersion in the vehicle; however, this is not required.

Compositions known in the art, preferably hypoallergenic and pH-controlled are especially preferred for topical administration, and include toilet waters, packs, lotions, skin milks or milky lotions. The preparations contain, besides the Conditioned medium and, optionally, other active ingredients, components usually employed in such preparations. Examples of such components are oils, fats, waxes, surfactants, humectants, thickening agents, antioxidants, viscosity stabilizers, chelating agents, buffers, preservatives, perfumes, dyestuffs, lower alkanols, and the like.

Examples of oils include fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearite, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants include butylated hydroxytoluene, butylated hydroxyanisole, propyl gallate, citric acid and ethoxyquin; examples of chelating agents include disodium edetate and ethanehydroxy diphosphate; examples of buffers include citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. These substances are merely exemplary, and those of skill in the art will recognize that other substances may be substituted with no loss of functionality.

For preparing compositions for topical administration, the concentration of conditioned medium may be more than about 0.00001, 0.00005, 0.0001, 0.001, 0.01, 0.1, 1, 5 or 10% by weight. In some embodiments, the concentration of the conditioned medium is more than about 0.00005%. The concentration of conditioned medium may be less than about 0.0001, 0.001, 0.001, 0.01, 0.1, 1, 5, 10 or 20% (all concentration percentages given herein are w/w unless otherwise indicated). In some embodiments, lower and higher concentrations are contemplated. In some embodiments employing ointments, lotions, or creams, the carrier for example, can contain 1 to 20%, in particular 5 to 15% of a humectant, 0.1 to 10% in particular from 0.5 to 5% of a thickener and water; or said carrier may consist of 70 to 99%, in particular 20 to 95% of a surfactant, and 0 to 20%, in particular 2.5 to 15% of a fat; or 80 to 99.9% in particular 90 to 99% of a thickener; or 5 to 15% of a surfactant, 2-15% of a humectant, 0 to 80% of an oil, very small (<2%) amounts of preservative, coloring agent and/or perfume, and water. In a toilet water, the carrier for example consists of 2 to 10% of a lower alcohol, 0.1 to 10% or in particular 0.5 to 1% of a surfactant, 1 to 20%, in particular 3 to 7% of a humectant, 0 to 5% of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume.

In some embodiments, conditioned medium, optionally with other active ingredients, is dissolved, mixed, or suspended in a mixture of emulsifying lanolin alcohols, waxes, and oils (e.g., EUCERIN™ Lotion) or a mixture of petrolatum or mineral oil, a quaternary ammonium compound, a fatty alcohol, and a fatty ester emollient (e.g., CUREL™ Lotion), or lotions that are substantially similar in composition.

EUCERIN™ Dry Skin Therapy Original Moisturizing Lotion comprises water, mineral oil, isopropyl myristate, PEG-40 sorbitan peroleate, glyceryl lanolate, sorbitol, propylene glycol, cetyl palmitate, magnesium sulfate, aluminum stearate, lanolin alcohol, BHT, methylchloroisothiazolinone, and methylisothiazolinone. CUREL™ Fragrance Free Daily Moisturizing Lotion comprises water, glycerin, distearyldimonium chloride, petrolatum, isopropyl palmitate, cetyl alcohol, dimethicone, sodium chloride, methylparaben, and propylparaben.

Some embodiments of compositions of the invention comprise conditioned medium in a lotion comprising a mixture of emulsifying lanolin alcohols, waxes, and oils (e.g., EUCERIN™ Dry Skin Therapy Original Moisturizing Lotion) at a concentration greater than about 0.00005%. Some embodiments of compositions of the invention comprise conditioned medium in a lotion comprising a mixture of emulsifying lanolin alcohols, waxes, and oils (e.g., EUCERIN™ Dry Skin Therapy Original Moisturizing Lotion) at a concentration of about 0.0001% to about 0.01%.

Some embodiments of compositions of the invention comprise conditioned medium in a lotion comprising a mixture of petrolatum or mineral oil, a quaternary ammonium compound, a fatty alcohol, and a fatty ester emollient (e.g., CUREL™ Fragrance Free Daily Moisturizing Lotion) at a concentration greater than about 0.00005%. Some embodiments of compositions of the invention comprise a Conditioned medium in a lotion comprising a mixture of petrolatum or mineral oil, a quaternary ammonium compound, a fatty alcohol, and a fatty ester emollient (e.g., CUREL™ Fragrance Free Daily Moisturizing Lotion) at a concentration of about 0.0001% to about 0.01%.

In addition, conditioned medium and, optionally, other active ingredients, may be formulated in liposome-containing compositions. Liposomes are artificial vesicles formed by amphipathic molecules such as polar lipids, for example, phosphatidyl cholines, ethanolamines and serines, sphingomyelins, cardiolipins, plasmalogens, phosphatidic acids and cerebrosides. Liposomes are formed when suitable amphipathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers.

The incorporation of conditioned medium into liposomal preparations suitable for topical application can be achieved by a number of methods. With respect to liposomal preparations, any known methods for preparing liposomes for treatment of a condition may be used. See, for example, Bangham et al., J. Mol. Biol, 23: 238-252 (1965) and Szoka et al., Proc. Natl Acad. Sci. 75: 4194-4198 (1978). Ligands may also be attached to the liposomes to direct these compositions to particular sites of action.

Liposomes containing conditioned medium and, optionally, other ingredients can be employed directly or they can be employed in a suitable pharmaceutically acceptable carrier for topical administration. The viscosity of the liposomes can be increased by the addition of one or more suitable thickening agents such as, for example xanthan gum, hydroxypropyl cellulose, hydroxypropyl methyl cellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. Exemplary metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purpose.

The organic component consists of a suitable non-toxic, cosmetically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatidylserine, phosphatidylethanolamine, phosphatidylinositol, lysophosphatidylcholine and phosphatidyl glycerol, for example. Other lipophilic additives may be employed in order to selectively modify the characteristics of the liposomes. Examples of such other additives include stearylamine, phosphatidic acid, tocopherol, cholesterol and lanolin extracts. In addition, other ingredients which can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Although conditioned medium is generally capable of penetrating cell membranes and reaching the deep layers of skin, it may be useful in some embodiments to also include a penetration enhancer in the formulations of the invention. A penetration enhancer is a substance that improves cutaneous penetration of a bioactive substance. Suitable penetration enhancers include, for example, dimethyl sulfoxide (DMSO), DMSO-like compounds, ethanolic compounds, pyroglutamic acid esters and other solvents or compounds known to those skilled in the pharmaceutical art which facilitate dermal penetration of the drugs or chemicals chosen for the pharmaceutical composition. Other penetration enhancers include amphiphiles such as L-amino acids, anionic surfactants, cationic surfactants, amphoteric surfactants, nonionic surfactants, fatty acids and alcohols. Additional penetration enhancers are disclosed in Remington: The Science and Practice of Pharmacy, 19th Edition (1995) on page 1583. The penetration enhancer chosen and the relative proportion of the penetration enhancer with respect to the active drugs or chemicals depends on the desired rate of delivery of the drugs or chemicals into the skin, which in turn depends on the condition being treated and the outcome sought. More specifically, the type and amount of enhancer is chosen so that a sufficiently high concentration of active drugs or chemicals is attained in the skin to treat the condition within the time period considered desirable.

Apart from the above-described compositions, use may be made of covers, e.g. plasters, bandages, dressings, gauze pads, patches and the like, containing an appropriate amount of conditioned medium and, optionally, other ingredients. In some cases use may be made of plasters, bandages, dressings, gauze pads, patches and the like which have been impregnated with a topical formulation containing the therapeutic formulation.

Additional Cosmetic and Dermatological Agents. Other cosmetic or dermatological agents may be included in methods and formulations of the invention. A “cosmetic or dermatological agent” as used herein, includes any substance whose administration for treatment of a cosmetic condition or to achieve a desired cosmetic effect, results in an status of the condition that is better than the status that would exist without the use of the cosmetic or dermatological agent.

Anti-wrinkling agents are one form of cosmetic or dermatological agents. Anti-skin wrinkling agents include a variety of agents, often in combination, that prevent or treat wrinkling through a variety of actions. Many approaches are taken to reduce the appearance of facial and other wrinkles based on the understanding of the molecular basis of wrinkle formation. Such treatments include cosmetic products, drug therapy and surgical procedures. For example, many cosmetic products contain hydroxy acids, which may stimulate collagen synthesis. Another common treatment utilizes retinol, retinoic acid, retinol palmitate, a derivative of vitamin A, (or prescribed versions, Retin-A and Renova) which may directly or indirectly stimulate collagen production or retard collagen degradation. Bicyclic aromatic compounds with retinoid-type activity, which are useful in particular in preventing or treating various keratinization disorders, are described in EP 679 630. These compounds are particularly active for repairing or combating chronological or actinic ageing of the skin, for example such as in anti-wrinkle products. Antioxidants such as vitamin C and E and coenzyme Q-10 are believed to counteract free radicals, which damage cells and cause aging and have been used in treatments of wrinkles. Recently, the FDA approved cosmetic use of Botox (an extremely purified form of botulinum toxin) to treat glabella frown lines.

Thus cosmetic or dermatological agents that complement cosmetic treatment of skin with the methods or compositions of the invention include, alone or in combination, the bicyclic aromatic compounds defined above, other compounds which have retinoid-type activity, free-radical scavengers, hydroxy or keto acids or derivatives thereof.

The term “free-radical scavenger” refers to, for example, .alpha.-tocopherol, superoxide dismutase, ubiquinol (e.g., coenzyme Q10) or certain metal-chelating agents. Hydroxy acids include, e.g., alpha-hydroxy acids such as lactic acid and glycolic acid or beta-hydroxy acids such as salicylic acid and salicylic acid derivatives such as the octanoyl derivative; other hydroxy acids and keto acids include malic, citric, mandelic, tartaric or glyceric acids or the salts, amides or esters thereof.

Other anti-wrinkling agents and anti-skin aging agents useful in the invention include sulfur-containing D and L amino acids and their derivatives and salts, particularly the N-acetyl derivatives, a preferred example of which is N-acetyl-L-cysteine; thiols, e.g. ethane thiol; fat-soluble vitamins, ascorbyl palmitate, ceramides, pseudoceramides (e.g., pseudoceramides described in U.S. Pat. Nos. 5,198,210; 4,778,823; 4,985,547; 5,175,321, all of which are incorporated by reference herein), phospholipids (e.g., distearoyl lecithin phospholipid), fatty acids, fatty alcohols, cholesterol, plant sterols, phytic acid, lipoic acid; lysophosphatidic acid, and skin peel agents (e.g., phenol and the like), and mixtures thereof. Preferred fatty acids or alcohols are those that have straight or branched alkyl chains containing 12-20 carbon atoms. A particularly preferred fatty acid is linoleic acid since linoleic acid assists in the absorption of ultraviolet light and furthermore is a vital component of the natural skin lipids. Other non-limiting examples of suitable anti-wrinkle actives for use herein are described in U.S. Pat. No. 6,217,888, which description is incorporated herein by reference.

Compositions for cosmetic treatment of skin may further include sunscreens to lower skin's exposure to harmful UV rays. Sunscreens include those materials commonly employed to absorb or block ultraviolet light. Illustrative compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, PARSOL MCX and BENZOPHENONE-3, respectively. Dermascreen may also be used.

Many other sunscreens are known to those of skill in the art. In some embodiments, sunscreens are FDA-approved or approved for use in the European Union. For example, FDA-approved sunscreens may be used, singly or, preferably, in combination. See, e.g., U.S. Pat. Nos. 5,169,624; 5,543,136; 5,849,273; 5,904,917; 6,224,852; 6,217,852; and Segarin et al., chapter Vil, pages 189 of Cosmetics Science and Technology, and Final Over-the-Counter Drug Products Monograph on Sunscreens (Federal Register, 1999:64:27666-27963), all of which are incorporated herein by reference. The exact amount of sunscreen employed in-the compositions can vary depending upon the degree of protection desired from the sun's UV radiation.

Cosmetic compositions of the invention may further include anti-acne agents. Anti-acne agents include benzoyl peroxide, antibiotics, e.g., erythromycin, clindamycin phosphate, 5,7-dichloro-8-hydroxyquinoline, resorcinol, resorcinol acetate, salicylic acid, azaleic acid, long chain dicarboxylic acids, sulfur, zinc, retinoids, antiandrogens, and various natural agents such as those derived from green tea, tea tree oil, and mixtures thereof. Other non-limiting examples of suitable anti-acne agents for use herein are described in U.S. Pat. No. 5,607,980, which description is incorporated herein by reference.

Other cosmetic and dermatological agents include anticellulite agents. Anticellulite agents include isobutylmethylxanthine, caffeine, theophylline, theobromine, aminophylline, yohimbine, and mixtures thereof.

Yet other cosmetic or dermatological agents that complement cosmetic treatment of skin include .alpha.-interferon, estradiol; progesterone; pregnanalone; methylsolanomethane (MSM); copper peptide (copper extract); plankton extract (phytosome); broparoestrol; estrone; adrostenedione; androstanediols; etc.

The compositions of the present invention may contain a wide range of additional components. The CTFA Cosmetic Ingredient Handbook, Seventh Edition, 1997 and the Eighth Edition, 2000, which are incorporated by reference herein in their entirety, describes a wide variety of ingredients commonly used in skin care compositions, which are suitable for use in the compositions of the present invention. Other topically-applied compounds are listed in Remington's Pharmaceutical Sciences, 20th Ed., Lippincott Williams & Witkins, Baltimore, Md. (2000) (hereinafter Remington's), U.S. Pharmacopeia and National Formulary, The United States Pharmacopeial Convention, Inc., Rockville, Md. and Physician's Desk Reference, Medical Economics Co., Inc., Oradell, N.J. incorporated herein by reference. The concentration of the other active ingredient in formulations provided by the invention is that which provides an effective amount of the other active ingredient; these concentrations are well-known in the art. See, e.g., the above references, as well as Textbook of Dermatology, Champion, Burton, Burns, and Bretnach, eds., Blackwell Publishing, 1998.

Discussions of cosmetic- and cosmeceutically-acceptable ingredients and formulations may also be found in, among other places, FDA Cosmetics Handbook, U.S. Food and Drug Administration; Handbook of Cosmetic and Personal Care Additives, Ash and Ash, compilers, 1994, Chemical Publishing, New York, N.Y.; Bennett's Cosmetic Formulary, 1993, Chemical Publishing Co.; Harry's Cosmeticology, 7.sup.th ed., Wilkinson & Moore, 1982 and 8.sup.th ed., Rieger, 2000, Chemical Publishing; Cosmetic Bench Reference-2001, Allerud Publishing Corp.; CTFA Compendium of Cosmetic Ingredient Composition, Nikitakis and McEwen, eds., 1990, Cosmetic, Toiletry, and Fragrance Association, Washington, D.C., Surfactant Encyclopedia, 2.sup.nd revised edition, Rieger, 1996, Allured Publishing; The Chemistry and Manufacture of Cosmetics, 2.sup.nd ed., De Navarre, Van Nostrand, Princeton, N.J.; Encyclopedia of Common Natural Ingredients Used in Food, Drugs, and Cosmetics, Leung, 1996, John Wiley; A Consumer's Dictionary of Cosmetic Ingredients, 5.sup.th ed., Winter, 1999, Three Rivers Press, New York, N.Y.; Cosmeceuticals: Active Skin Treatment, 1998, Allured Publishing; Handbook of Cosmetic Science and Technology, Knowlton and Pearce, 1993, Elsevier Advanced Technology, Oxford, UK; Personal-Care Formulas, 1997, Allured Publishing; Beginning Cosmetic Chemistry, Scheuller and Romanowski, 1999, Allured Publishing; and Skin Permeation: Fundamentals and Application, Zatz, 1993, Allured Publishing. Discussions of pharmaceutically-acceptable ingredients and formulations may be found in, among other places, Remington's Pharmaceutical Sciences, 18th ed., Gennaro, ed., 1990, Mack Publishing.

Kits

In still another aspect, the present invention provides kits for the cosmetic treatment of skin or to produce a desired cosmetic result. These kits comprise conditioned medium or any of the conditioned medium-containing compositions described herein, in a container or containers which are held in suitable packaging. In some embodiments the kits further contain instructions teaching the use of the kit according to the various methods and approaches described herein. Such kits may also include information, such as scientific literature references, package insert materials, cosmetic trial results, and/or summaries of these and the like, which indicate or establish the activities and/or advantages of the composition. Such information may be based on the results of various studies, for example, studies using experimental animals involving in vivo models and studies based on human cosmetic or clinical trials. Kits described herein can be provided, marketed and/or promoted to health care providers (e.g., dermatologists and other physicians), skin care appearance care providers, including cosmetologists, hair stylists, and the like. Kits for cosmetic use may also be provided, marketed and/or promoted directly to consumers. Kits may be marketed in spas and retail outlets.

All patents and patent applications, publications, scientific articles, and other referenced materials mentioned in this specification are indicative of the levels of skill of those skilled in the art to which the invention pertains, and each of which is hereby incorporated by reference to the same extent as if each reference had been incorporated by reference in its entirety individually. Applicants reserve the right to physically incorporate into this specification any and all materials and information from any such patents and patent applications, publications, scientific articles, electronically available information, and other referenced materials or documents.

The specific media compositions, culture systems, cosmetic compositions and methods of making and using described in this specification are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably may be practiced in the absence of any element or elements, or limitation or limitations, which is not specifically disclosed herein as essential. Also, the terms “comprising”, “including”, “containing”, etc. are to be read expansively and without limitation. It must be noted that as used herein and in the appended claims, the singular forms “a”, “an”, and “the” include plural reference, and that the use of the word “or,” for example, as in the case of “a or b” may refer to a alone, to b alone, or to a and b together, unless the context clearly dictates otherwise.

The terms and expressions that have been employed are used as terms of description and not of limitation, and there is no intent in the use of such terms and expressions to exclude any now-existing or later-developed equivalent of the features shown and described or portions thereof, but it is recognized that various modifications are possible within the scope of the invention as claimed. Thus, it will be understood that although the present invention has been specifically disclosed by preferred embodiments and optional features, modification and/or variation of the disclosed elements may be resorted to by those skilled in the art, and that such modifications and variations are within the scope of the invention as claimed.

### REFERENCES

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A composition for administration to skin of an individual, the composition comprising an amount of a medium conditioned by growth of embryonic stem cells that is effective to provide an improvement in the health or appearance of the skin of the individual and a suitable vehicle.

2. The composition of claim 1 wherein the embryonic stem cells are human embryonic stem cells.

3. The composition of claim 2 that is substantially free from non-human animal products.

4. The composition of claim 1 wherein the improvement in the appearance or health of the skin of the individual is an improvement in a member of the group consisting of wrinkling, folds, sagging, age spots, uneven pigmentation, thinning, elasticity, scarring, surface roughness, surface roughness, surface vessels, redness, and pore size.

5. A method of treating skin of an individual in need thereof comprising administering to the skin of the individual a composition comprising an amount of a medium conditioned by growth of human embryonic stem cells that is effective to provide an improvement in the appearance or health of the skin of the individual and a suitable vehicle.

6. The method of claim 5 wherein the embryonic stem cells are human embryonic stem cells.

7. The method of claim 6 wherein the composition is substantially free from non-human animal products.

8. The method of claim 5 wherein the improvement in the appearance or health of the skin of the individual is an improvement in a member of the group consisting of wrinkling, folds, sagging, age spots, uneven pigmentation, thinning, elasticity, scarring, surface roughness, surface roughness, surface vessels, redness, and pore size.

9. A method of making a composition for treatment of skin of an individual in need thereof comprising combining an amount of a medium conditioned by growth of embryonic stem cells that is effective to provide an improvement in the appearance or health of the skin of the individual and a suitable vehicle.

10. The method of claim 9 wherein the embryonic stem cells are human embryonic stem cells.

11. The method of claim 10 wherein the composition is substantially free from non-human animal products.

12. A use of a medium conditioned by growth of embryonic stem cells to prepare a medicament for administration to skin of an individual in need thereof.

13. A kit comprising a composition comprising: (a) composition for administration to skin of an individual, the composition comprising an amount of a medium conditioned by growth of embryonic stem cells that is effective to provide an improvement in the appearance or health of the skin of the individual and a suitable vehicle; and (2) suitable packaging.

14. The kit of claim 13 wherein the embryonic stem cells are human embryonic stem cells.

15. The kit of claim 14 wherein the composition is substantially free from non-human animal products.
